(19) United States
(12) Patent Application Publication
(10) Pub. No.: US 2012/0189546 A1
(43) Pub. Date: Jul. 26, 2012

(54) Radiolabelling Method Using Cycloalkyl Groups

(75) Inventors: Keith Graham, Berlin (DE); Heribert Schmitt-Willich, Berlin (DE); Dominic Franck, Berlin (DE)

(73) Assignee: BAYER PHARMA AKTIENGESELLSCHAFT, Berlin (DE)

(21) Appl. No.: 13/383,228

(22) PCT Filed: Jul. 9, 2010
(86) PCT No.: PCT/EP2010/004199

§ 371 Date: Apr. 4, 2012

(30) Foreign Application Priority Data
Jul. 11, 2009 (EP) 09075293.2

(51) Int. Cl. A61K 51/04; C07C 35/04; C07C 229/36; C07D 487/04; C07D 241/04; C07C 271/18; C07C 61/04; C07C 309/73

(52) U.S. Cl. 424/1.89; 562/505; 568/839; 558/57; 544/281; 544/390; 560/27; 562/444

(57) ABSTRACT

This invention relates to novel cyclo alkyl compounds suitable for labeling by ¹⁸F, methods of preparing such a compound, compositions comprising such compounds, kits comprising such compounds or compositions and uses of such compounds, compositions or kits for diagnostic imaging by positron emission tomography (PET).

### FIELD OF INVENTION

This invention relates to novel compounds suitable for labeling by ¹⁸F, methods of preparing such a compound, compositions comprising such compounds, kits comprising such compounds or compositions and uses of such compounds, compositions or kits for diagnostic imaging by positron emission tomography (PET).

### BACKGROUND

Molecular imaging has the potential to detect disease progression or therapeutic effectiveness earlier than most conventional methods in the fields of oncology, neurology and cardiology. Of the several promising molecular imaging technologies having been developed as optical imaging and MRI, PET is of particular interest for drug development because of its high sensitivity and ability to provide quantitative and kinetic data.

Positron emitting isotopes include carbon, nitrogen, and oxygen. These isotopes can replace their non-radioactive counterparts in target compounds to produce tracers that function biologically and are chemically identical to the original molecules for PET imaging. On the other hand, ¹⁸F is the most convenient labeling isotope due to its relatively long half life (109.6 min) which permits the preparation of diagnostic tracers and subsequent study of biochemical processes. In addition, its low β+ energy (635 keV) is also advantageous.

The aliphatic ¹⁸F-fluorination reaction is of great importance for ¹⁸F-labeled radiopharmaceuticals which are used as in vivo imaging agents targeting and visualizing diseases, e.g. solid tumours or diseases of the brain. A very important technical goal in using ¹⁸F-labeled radiopharmaceuticals is the quick preparation and administration of the radioactive compound due to the fact that the ¹⁸F isotopes have a short half-life of about only 110 minutes.

Using radiolabeled imaging agents in molecular imaging, in particular PET, can have a number of drawbacks:
1. The position of the radiolabel, introduced either directly or via indirect, so named prosthetic groups, can be metabolically unstable, thus, giving rise to radiolabeled metabolites which can potentially interfere with the image quality.
2. Introducing a radiolabel via a prosthetic group to a biomolecule via conjugation methods can alter the pharmacokinetics and behaviour of the conjugated biomolecule due to a number of factors including increased lipophilicity.

The metabolism of radiolabel imaging agents, in particular PET imaging agents, has been well-documented in the literature. In Scheme 1 are some examples of PET tracers known to undergo metabolism, [¹¹C]SCH23390 (De Jesus et al., J. Radioanalytical Nucl. Chem., 1988, 125, 65-73), [¹⁸F]FFMZ (Chang et al., Nucl. Med. Bio., 2005, 32, 263-268), [¹⁸F]FE-SA4503 (Kawamura et al., Nucl. Med. Bio., 2003, 30, 273-284, Elsinga et al., Synapse, 2002, 43, 259-267), S—[¹¹C]SME-IMPY, N—[¹¹C]SME-IMPY (Cai et al., J. Med. Chem., 2008, 51, 148-158), [¹⁸F]FET (Langen et al., Nucl. Med. Biol., 2006, 33, 287-294), [¹⁸F]FETO (Ettlinger et al., Eur. J. Nucl. Med. Mol. Imaging, 2006, 33, 928-931 and references cited within) and [¹⁸F]FEOBV (Mulholland et al., Synapse, 1998, 30, 263-274).

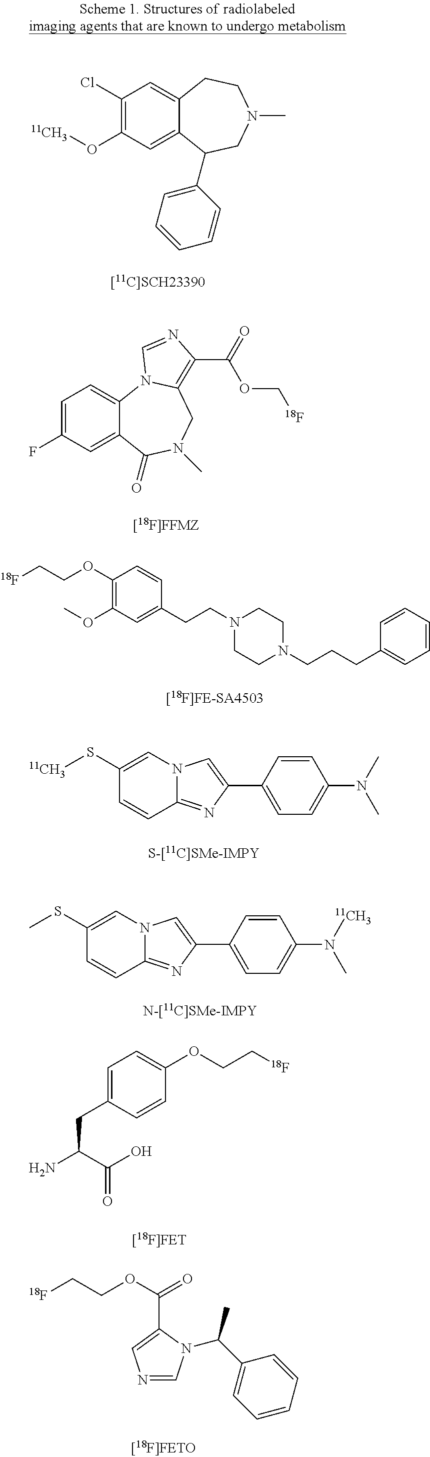

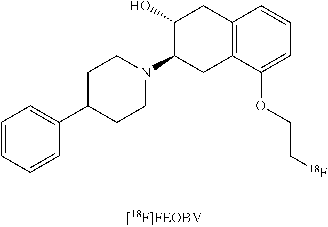

The examples in Scheme 1 highlight that radiolabeled alkyl and fluoroalkyl chains attached to heteroatoms, e.g. oxygen, nitrogen and sulfur, undergo metabolism to a large extent. For the [¹⁸F]fluoroethoxy groups the first majority metabolite is believed to [¹⁸F]fluoroethanol. The metabolism of fluoroethanol has already been reported (Treble, Biochemistry, 1962, 82, 129-134). Therefore the [¹⁸F]fluoroethanol will be further metabolized via different biological pathways, e.g. oxidation and the citric acid cycle, to give [¹⁸F]fluoroactealdehyde, [¹⁸F]fluoroacetate and [¹⁸F]fluorocitrate. These metabolites will then behave differently in the body and can cause considerable background noise which will ultimately give a poorer image quality in comparison to radiolabeled imaging agent where the site of the labeling is more metabolically stable.

In the literature it has been reported that heteroatoms substituted with cycloalkyl rings can be more metabolically stable than when substituted with an alkyl group. This was the case for Serotonin 5-HT_{1A} aminopyrimidine partial agonists, when the cyclopropyl group substituted on a nitrogen was replaced by bulkier alkyl groups the stability in human liver microsomes decreased (Dounay et al., Bioorg. Med. Chem. Lett., 2009, 19, 1159-1163). This has also been shown for 11-β-hydroxysteroid dehydrogenase 1 (11-β-HSD-1) inhibitors, when the nitrogen was substituted with cycloalkyl rings they were more metabolically stable in mouse liver microsomes than the alkyl or bulky alkyl counterparts (Sorensen et al., Bioorg. Med. Chem. Lett., 2006, 16, 5958-5962). Other examples of improved stability via cycloalkyl groups include PDE4 inhibitors (Chauret et al., Bioorg. Med. Chem. Lett., 2002, 12, 2149-2152) and NK1 selective antagonists (Bioorg. Med. Chem. Lett., 2006, 16, 3859-3863).

The radiolabeling of the majority of biomolecules, particularly the larger biomolecules, e.g. peptides, single-chain fragments, antibodies and aptamers, is carried out via ‘indirect methods’ whereby a prosthetic group or synthon, containing a defined reactive moiety, is first synthesized and then subsequently conjugated to a defined functional group(s) within the biomolecule of interest. These conjugations conditions are preferably carried out in aqueous media and under mild conditions. The most common conjugations using radiolabeled prosthetic groups have the radiolabel attached to an aromatic ring, e.g. [¹²⁵I] Bolton-Hunter's reagent, [¹⁸F]SFB, [¹⁸F]FBCHO, [¹⁸F]FPB and [¹⁸F]FBAM (Scheme 2).

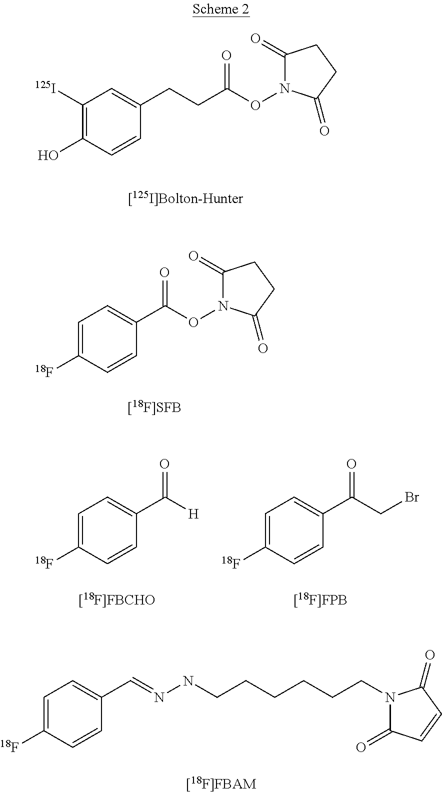

The aromatic carbon-fluorine bond is typically very stable in vivo, however, the addition of this benzene ring will add lipophilicity to the biomolecule of interest and thus alter the biological characteristics of the compound, e.g. binding affinity, biodistribution etc. This point is reitified by the following statement from Wester and Schottelius “Although resulting in products with a somewhat higher lipophilicity, the 4-[¹⁸F]fluorobenzoyl moiety has been extensively used for peptide labeling.” (PET Chemistry—The Driving Force in Molecular Imaging, Ernst Schering Foundation Symposium Proceedings Vol. 64. Chapter 4, 79-111, Springer Berlin Heidelberg, Eds. Schubiger, Friebe and Lehmann). Other non-aromatic prosthetic groups tend to contain aliphatic carbon chains with the [¹⁸F]fluorine atom in a primary position, which is again more prone to in vivo metabolism/defluorination.

There are numerous publications reporting the increased lipophilicity of biomolecules conjugation with aromatic prosthetic groups, i.e. αvβ6 specific peptides with [¹⁸F]SFB (Hausner et al., J. Med. Chem., 2008, 51, 5901-5904), Neurotensin(8-13) peptides analogs with [¹⁸F]SFB (Bergmann et al., Nucl. Med. Bio. 2002, 29, 61-72), chemotactic hexapaptide with [¹³¹I]SIB (Pozzi et al., Appl. Radiat. Isot., 2006, 64, 668-676), LTB4 antagonists with [¹⁸F]FBCHO (Rennen et al., Nucl. Med. Biol., 2007, 34, 691-695), Octreotide (Guhlke et al., Nucl. Med. Biol., 1994, 21, 819-825), αvβ3 (Haubner et al., J. Nucl. Med., 1999, 40, 1061).

In the literature, unnatural α-cyclic amino acids, in particular aminocyclopentanecarboxlic (ACPC), are known to inhibit tumour growth (Connors et al., Biochem. Pharmacol. 1960, 5, 108-129; Martel et al., Can. J. Biochem. Physiol., 1959, 37, 433-439). These amino acids have been radiolabeled, mainly with PET isotopes, and have been explored as tumour imaging agents. These PET labeled cyclic amino acids have been labeled with both C-11 and F-18, as illustrated in Scheme 3, i.e. [¹¹C]ACBC (Washburn et al., J. Nucl. Med., 1979, 20, 1055-1061) [¹¹]ACPC (Washburn et al., Cancer Res., 1978, 38, 2271-2273), 3-[¹⁸F]anti-FACBC (Shoup et al., J. Nucl. Med., 1999, 40, 331), 3-[¹⁸F]syn-FACBC (Yu et al., Bioorg. Med. Chem., 2009, 17, 1982-1990) and 2-[¹⁸F]FACPC (WO2007/001958A2). These are the only examples with a PET radioisotope incorporated into a cycloalkyl ring.

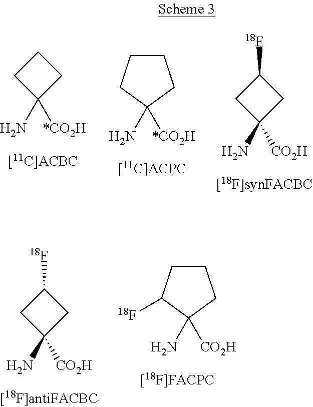

Although these radiolabeled cyclic amino acids are known, the use of radiolabeled cyclic alkyl rings have not been explored as metabolically stable groups that can be incorporated in biomolecules of interest.

### SUMMARY OF THE INVENTION

In present invention relates to the use of fluorocycloalkyl rings for increasing the stability of substance, in particular the metabolic stability. Preferably, the invention relates to increasing stability of substance containing a radioisotope. The preferred radioisotope would be a radiohalogen, the most preferred radiohalogen would be fluorine-18. Scheme 4 refers to the method for increasing the stability of the radiolabel, particularly in position where metabolism is likely to occur.

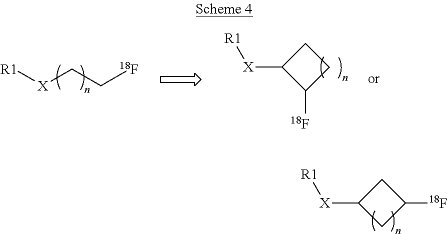

Another aspect of the current invention is the use of fluorocycloalkyl rings as synthons that can be conjugated to biomolecules of interest. The clogP values of a fluorocyclobutyl carboxylic acid and a fluorocyclobutyl aldehyde are compared with their analogous aromatic derivatives (Scheme 5). It is clear to see that there is a considerable difference in the clogP values between the aromatic and cyclobutyl analogues for both the carboxylic acid (A=+1.72) and aldehyde (Δ=+1.38) synthons.

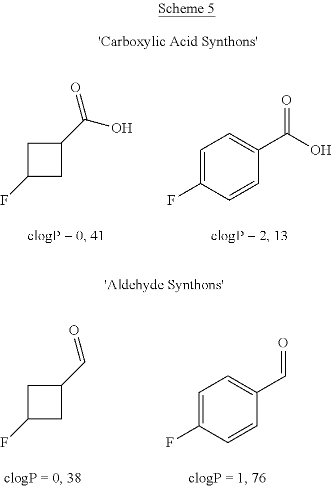

When comparing the clogP values for a native Arginine-Glycine-Aspartate (RGD) peptide, [Dab-RGDF], with the cyclobutyl carboxylic acid conjugated derivates, [Dab(3-fluorocyclobutanoyl)-RGDF] (Dab=2,4.diaminobutyne acid), and the benzoic acid derivative, [Dab(4-fluorobenzoyl)-RGDF], it is clear to see that the difference in clogP is +1.78 (Scheme 6). This additional lipophilicity for the benzoylated derivative will also changed to pharmacokinetic profile of the peptide.

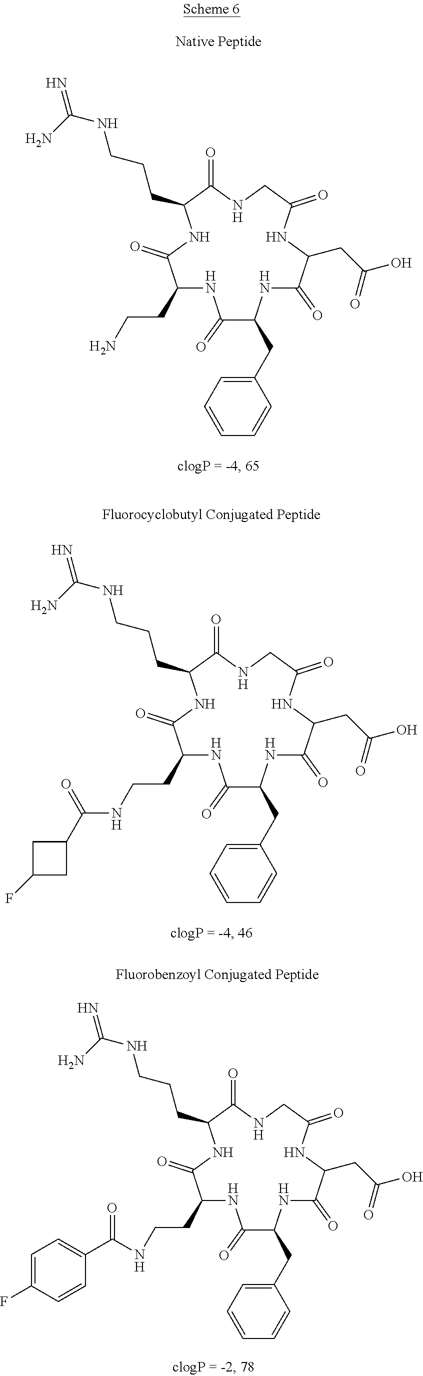

The same holds true when one compares the native peptide against the conjugated cyclobutyl aldehyde and the benzaldehyde (Scheme 6), the difference in clogP is also +1.49 for the benzoylated peptide—this significant lipophilic difference will again influence the pharmacokinetic profile of the peptide.

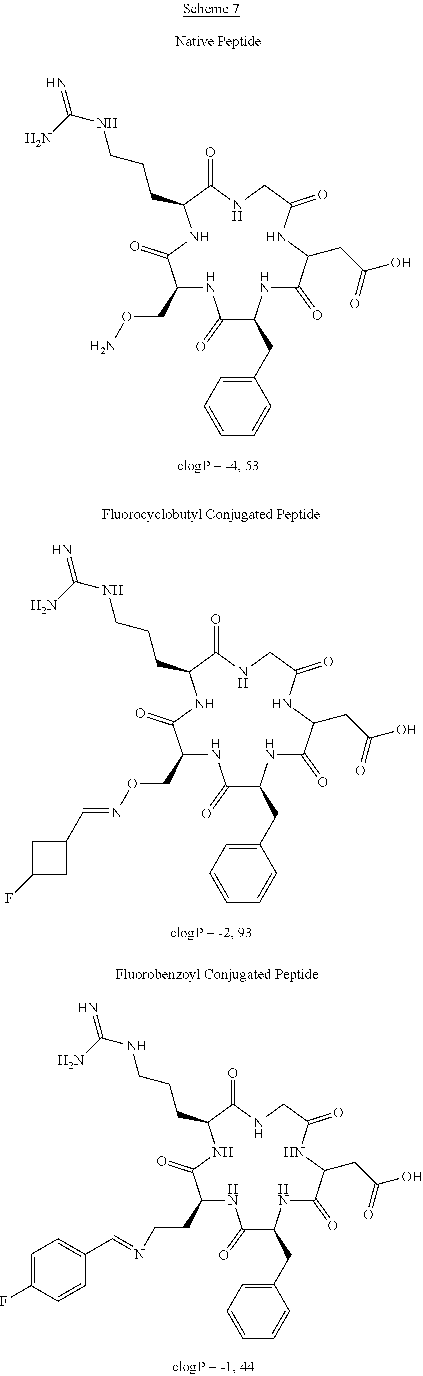

### DRAWINGS

FIG. 1: Chromatogram (radio trace) of purified toluene-4-sulfonic acid 3-[18F]fluoro-cyclobutyl ester (31).

FIG. 2: Chromatogram (radio trace) of purified (S)-2-Amino-3-[4-(3-[¹⁸F]fluoro-cyclobutoxy)-phenyl]-propionic acid (32) compared to the cold reference.

FIG. 3: Chromatogram (radio trace) of reaction mixture of Methyl N-(tert-butoxycarbonyl)-O-(cis-3-fluorocyclobutyl)-L-tyrosinate (33).

FIG. 4: Chromatogram (radio trace) of (cis)-benzyl 3-18F])fluorocyclobutanecarboxylate (35).

FIG. 5: Chromatogram (radio trace) of 3-[18F]fluorocyclobutanecarboxylate (36).

FIGS. 6 and 7: Uptake of compound 29 in A549 human lung carcinoma cell line.

FIG. 8: Uptake of the radiolabeled [¹⁸F] compound 32b into A549 cells.

FIG. 9: Competition experiment and uptake of the radiolabeled compound 32b ([18F] labeled) into A549 cells.

### DETAILED DESCRIPTION OF THE INVENTION

In a first aspect the invention relates to novel compounds of Formula I suitable for labeling with a radioisotope.

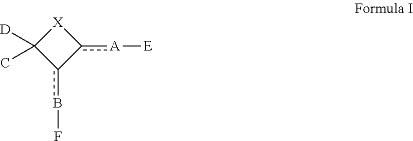

wherein

### A=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### B=H, —O—, ═O, S, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, O or S,

### C=H, Leaving Group (LG), or R′,

### D=H, Leaving Group (LG), or R′,

E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein W is a linker and Z is a targeting agent or vector,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein W is a linker and Z is a targeting agent or vector,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)₆, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2,
when A or B is ═O then E or F is absent
and pharmaceutical salt, diastereomere and enantiomere thereof.

Compounds of Formula I are optionally protected at the functional entities of the invention compounds by protecting groups. Known protecting groups are alcohol-, amine-, aminoxy-, carbonyl-, carboxylic-, ketone-, aldehyde-, amino alcohol-, phosphate-protecting groups. Protecting groups which are known or obvious to someone skilled in the art and which are chosen from but not limited to those described in the textbook Greene and Wuts, Protecting groups in Organic Synthesis, fourth edition, included herewith by reference. A protected compound of Formula I is named compound of Formula Ia.

O-protecting group is selected from the group comprising Methyl, Ethyl, Propyl, Butyl and t-Butyl. Preferably, O-protecting group is selected from the group comprising Methyl, Ethyl and t-Butyl. More preferably, O-protecting group is t-Butyl.

N-protecting group is selected from the group comprising

Carbobenzyloxy (Cbz), tert-Butyloxycarbonyl (BOC), 9-Fluorenylmethyloxycarbonyl (FMOC), and Triphenylmethyl. Preferably, N-protecting group is selected from the group comprising Carbobenzyloxy (Cbz), tert-Butyloxycarbonyl (BOC) and 9-Fluorenylmethyloxycarbonyl (FMOC). More preferably, N-protecting group is tert-Butyloxycarbonyl (BOC) or 9-Fluorenylmethyloxycarbonyl (FMOC).

Preferably, A and B are independently from eachother H, —O—, ═O, —S—, ═S, N(R′), NYR′, C(R′)(R″), CR′R″, C(O), C(O)O, C(O)OR′ or C(O)R′R″.

More preferably, A is —O—, ═O, —S—, ═S, N(R′), NYR′, C(R′)(R″), CR′R″, C(O), C(O)O, C(O)OR′ or C(O)R′R″ and B is H.

More preferably, B is —O—, ═O, —S—, ═S, N(R′), NYR′, C(R′)(R″), CR′R″, C(O), C(O)O, C(O)OR′ or C(O)R′R″ and A is H.

Even more preferably, A is —O—, C(O), or C(O)O and B is H. Even more preferably, B is —O—, C(O), or C(O)O and A is H.

The Leaving Group (LG) is a suitable leaving group that can be replaced by a radioisotope atom. The Leaving Group (LG) is a leaving group known or obvious to someone skilled in the art and which is taken from but not limited to those described or named in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O-nonaflat”), Carey and Sundberg, Organische Synthese, (1995), page 279-281, table 5.8; or Netscher, Recent Res. Dev. Org. Chem., 2003, 7, 71-83, Scheme 1, 2, 10 and 15.

The Leaving Group (LG) is selected from the group comprising fluoro, chloro, bromo and iodo, mesyloxy, tosyloxy, trifluoromethylsulfonyloxy, nonafluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-tertbutyl-phenyl)sulfonyloxy and (4-methoxy-phenyl)sulfonyloxy.

Preferably, LG is selected from the group comprising iodo, bromo, chloro, mesyloxy, tosyloxy, (4-nitro-phenyl)sulfonyloxy and (2-nitro-phenyl)sulfonyloxy.

More preferably, LG is selected from the group comprising mesyloxy, tosyloxy, trifluoromethylsulfonyloxy and (4-nitro-phenyl)sulfonyloxy.

Preferably, when D is a Leaving Group (LG) then C is H.

Preferably, when C is a Leaving Group (LG) then D is H.

Preferably none of D or C is a Leaving Group (LG).

W is a Linker well known in the art that is suitable for binding a targeting agent or vector to a small entity.

Preferably, W is selected but not limited to NR′, O, C(R′R″), branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, aminoacid, CO(CH₂)ₙ, [O(CH₂)ₙ—O(CH₂)ₙ]ₘ, or —O(CH₂)ₙ, [O(CH₂)ₙ—O(CH₂)ₙ]ₘ, n=1 to 6 and m=1 to 6.

The targeting agent or vector is typically selected from the group consisting of a synthetic small molecule, a pharmaceutically active compound (i.e., a drug molecule), a metabolite, a signaling molecule, an hormone, a peptide, a protein, a receptor antagonist, a receptor agonist, a receptor inverse agonist, a vitamin, an essential nutrient, an amino acid, a fatty acid, a lipid, a nucleic acid, a mono-, di-, tri- or polysaccharide, a steroid, and the like. It will be understood that some of the aforementioned options will overlap in their meaning, i.e., a peptide may for example also be a pharmaceutically active compound, or a hormone may be a signaling molecule or a peptide hormone. Furthermore, it will be understood that also derivatives of the aforementioned substance classes are encompassed.

The targeting agent or vector (or, optionally, any metabolite), is preferably a moiety that specifically binds to a target site in a mammalian body. Specific binding in this context means that the compound targeting agent or vector for that matter, accumulates to a larger extent at this target site compared to the surrounding tissues or cells. For example, the targeting agent or vector may specifically bind to a receptor or integrin or enzyme that is preferentially expressed at a pathologic site within the mammalian body, or the targeting agent or vector may be specifically transported by a transporter that is preferentially expressed at a pathologic site within the mammalian body. In some embodiments, the receptor, integrin, enzyme, or transporter is exclusively expressed at a pathologic site within the mammalian body, i.e., to sites that are different or absent in healthy subjects, or vice versa. In this context, it will be understood that the targeting agent or vector preferably binds specifically to a receptor/or integrin/or enzyme/or transporter that is exclusively expressed or present at a pathologic site within the mammalian body and not expressed or present at a non-pathologic site, although the latter is—while no doubt highly desirable—rarely achieved in practice.

Examples for specific binding include, but are not limited to, specific binding to a site of infection, inflammation, cancer, platelet aggregation, angiogenesis, necrosis, ischemia, tissue hypoxia, angiogenic vessels, Alzheimer's disease plaques, atherosclerotic plaques, pancreatic islet cells, thrombi, serotonin transporters, neuroepinephrin transporters, LAT 1 transporters, apoptotic cells, macrophages, neutrophils, EDB fibronectin, receptor tyrosine kinases, cardiac sympathetic neurons, and the like.

In preferred embodiments, targeting agent or vector may be selected from the group consisting of a synthetic small molecule, a pharmaceutically active compound (drug), a peptide, a metabolite, a signaling molecule, a hormone, a protein, a receptor antagonist, a receptor agonist, a receptor inverse agonist, a vitamin, an essential nutrient, an amino acid, a fatty acid, a lipid, a nucleic acid, a mono-, di-, tri-, or polysaccharide, a steroid, a hormone and the like. More specifically, the targeting agent or vector may be selected from the group consisting of glucose, galactose, fructose, mannitol, sucrose, or stachyose and derivatives thereof, glutamine, glutamate, tyrosine, leucine, methionine, tryptophan, acetate, choline, thymidine, folate, methotrexate, Arg-Gly-Asp (RGD) peptides, chemotactic peptides, alpha melanotropin peptide, somatostatin, bombesin, human pro-insulin connecting peptides and analogues thereof, GPIIb/IIIa-binding compounds, PF4-binding compounds, αvβ3, αvβ6, or α4β1 integrin-binding compounds, somatostatin receptor binding compounds, GLP-1 receptor binding compounds, sigma 2 receptor binding compounds, sigma 1 receptor binding compounds, peripheral benzodiazepine receptor binding compounds, PSMA binding compounds, estrogen receptor binding compounds, androgen receptor binding compounds, serotonin transporter binding compounds, neuroepinephrine transporter binding compounds, dopamine transporter binding compounds, LAT transporter binding compounds and hormones such as peptide hormones, and the like.

In a preferred embodiment, the compound of Formula I is a compound of Formula I wherein E=H, OR′, SR′, NR′, or CR′ₚ and F=H, OR′, SR′, NR′, or CR′ₚ named compound of Formula I*.

Preferably, E=absent, H, C(R′)(R″), CR′R″, or W-Z, wherein W is a linker and Z is a targeting agent or vector. More preferably, E=H, C(R′)(R″), CR′R″, or W-Z, wherein W is a linker and Z is a targeting agent or vector.

Preferably, E=absent, H, C(R′)(R″), or CR′R″.

Preferably, F=absent, H, C(R′)(R″), CR′R″, or W-Z, wherein W is a linker and Z is a targeting agent or vector. More preferably, F=H, C(R′)(R″), CR′R″, or W-Z, wherein W is a linker and Z is a targeting agent or vector.

Preferably, F=absent, H, C(R′)(R″), or CR′R″.

Preferably, R′=H, OH, NH, branched or linear C₁-C₆ alkyl, branched or linear O—C₁-C₆ alkyl, branched or linear C₁-C₆ alkoxy, substituted or unsubstituted aryl, preferably phenyl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,

wherein n=1 to 6 and m=1 to 6.

Preferably, n=1 to 3 or 4 to 6 and m=1 to 3 or 4 to 6.

Preferably, branched or linear C₁-C₆ alky is methyl, ethyl or butyl.

More preferably, R′=H, OH, methyl, ethyl or butyl.

Preferably, R″=H, OH, NH, branched or linear C₁-C₆ alkyl, branched or linear O—C₁-C₆ alkyl, branched or linear C₁-C₆ alkoxy, substituted or unsubstituted aryl, preferably phenyl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,

wherein n=1 to 6 and m=1 to 6.

Preferably, n=1 to 3 or 4 to 6 and m=1 to 3 or 4 to 6.

Preferably, branched or linear C₁-C₆ alky is methyl, ethyl or butyl.

More preferably, R″=H, OH, methyl, ethyl, butyl or phenyl.

Preferably, X=(CH₂)_{q} wherein q=1 or 2, preferably 1.

In a first embodiment, the invention relates to novel compounds of Formula I suitable for labeling with a radioisotope wherein the compounds are suitable for direct labeling.

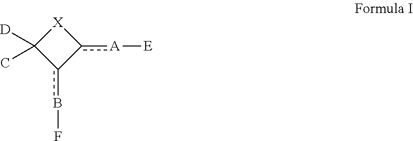

wherein

### A=H, —O—, ═O, —S—, ═S, N, N(R′), NYR′, P(R′)(R″), P(O)(R′)R″, C(R′)(R″), CR′R″, C(O), C(O)O, C(O)OR′ C(O)R′R″, SO, SO2, or SO2NR′,

### B=H, —O—, ═O, S, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, —O— or S,

### C=H, Leaving Group (LG), or R′,

### D=H, Leaving Group (LG), or R′,

E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein
W is a linker and Z is a targeting agent or vector,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein
W is a linker and Z is a targeting agent or vector,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2,
with the proviso that at least E or F is W-Z and
with the proviso that when A or B is ═O then E or F is absent,
and pharmaceutical or suitable salt, diastereomere and enantiomere thereof.

In a second embodiment, the invention relates to novel compounds of Formula I suitable for labeling with a radioisotope wherein the compounds are suitable for indirect labeling.

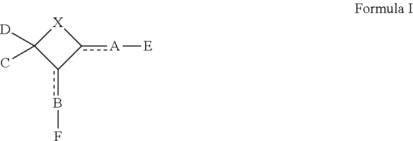

wherein

### A=H, —O—, ═O, —S—, ═S, N, N(R′), NYR′, P(R′)(R″), P(O)(R′)R″, C(R′)(R″), CR′R″, C(O), C(O)O, C(O)OR′ C(O)R′R″, SO, SO2, or SO2NR′,

### B=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, —O— or S, C═H, Leaving Group (LG), or R′,

### D=H, Leaving Group (LG), or R′,

E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, or SO2NR′,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, or SO2NR′,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2,
with the proviso that when en A or B is ═O then E or F is absent and
with the proviso that E and F cannot be absent at the same time,
and pharmaceutical or suitable salt, diastereomere and enantiomere thereof.

In a third embodiment, the invention relates to novel compounds of Formula I wherein

A=bond, —O—, —S—, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,
B=bond, —O—, —S—, N, N(R′), NYR′, P(R′)(R″), P(O)(R′)R″, C(R′)(R″), CR′R″, C(O), C(O)O, C(O)OR′, C(O)R′R″, SO, SO2, or SO2NR′.

Preferably, A and/or B is bond.

Embodiments and preferred features can be combined together and are within the scope of the invention.

Invention compounds are but not limited to

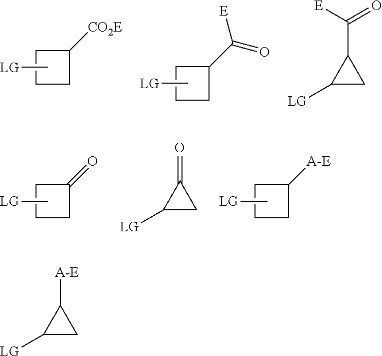

LG: Leaving group
E and A as disclosed above

### cis-Benzyl 3-(tosyloxy)cyclobutanecarboxylate

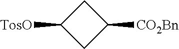

### cis-3-(Benzyloxy)cyclobutyl toluene-4-sulfonate

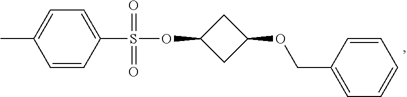

### trans-3-{3-[N-(2-{4-[4-(Benzyloxy)phenyl]piperazin-1-yl}-2-oxoethyl)carbamoyl]phenoxy}cyclobutyl toluene-4-sulfonate

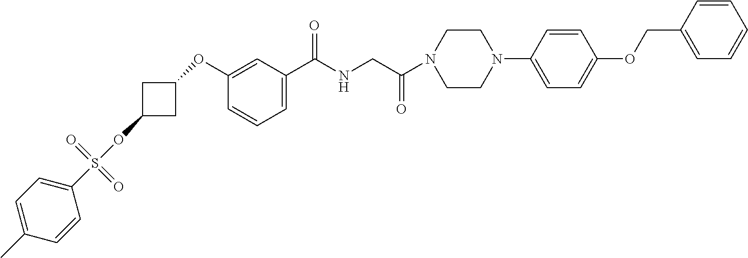

### Methyl N-(tert-butoxycarbonyl)-O-[trans-3-(tosyloxy)cyclobutyl]-L-tyrosinate

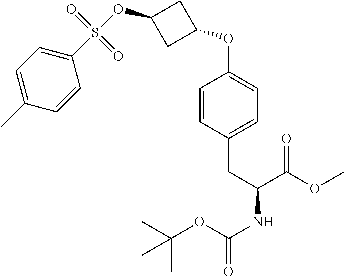

### Methyl N-(tert-butoxy carbonyl)-O-[cis-3-(tosyloxy)cyclobutyl]-L-tyros

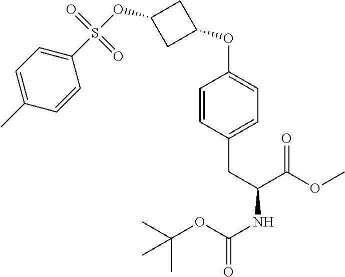

### cis-Methyl 3-(tosyloxy)cyclobutanecarboxylate

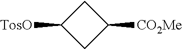

### cis-Cyclobutane-1,3-diyl bis(toluene-4-sulfonate

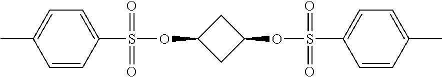

In a second aspect the invention relates to novel compounds of formula II.

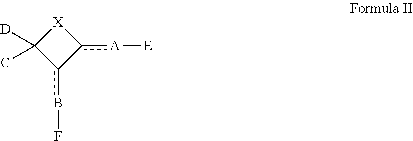

wherein

### A=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### B=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, O or S,

C=H, radioisotope, halogen or R′,
D=H, radioisotope, halogen or R′,
E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein
W is a linker and Z is a targeting agent,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein
W is a linker and Z is a targeting agent,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2
when A or B is ═O then E or F is absent
and pharmaceutical salt, diastereomere and enantiomere thereof.

W is a Linker well known in the art that is suitable for binding a targeting agent or vector to a small entity.

Preferably, W is selected but not limited to NR′, —O—, C(R′R″), branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, aminoacid, CO(CH₂)ₙ, [O(CH₂)ₙ—O(CH₂)ₙ]ₘ, or —O(CH₂)ₙ, [O(CH₂)ₙ—O(CH₂)ₙ]ₘ,

n=1 to 6 and m=1 to 6.

Compounds of Formula II are optionally protected at the functional entities of the invention compounds by protecting groups. Known protecting groups are alcohol-, amine-, aminoxy-, carbonyl-, carboxylic-, ketone-, aldehyde-, amino alcohol-, phosphate-protecting groups. A protected compound of Formula II is named compound of Formula IIa.

Preferably, A and B are independently from eachother H, —O—, ═O, —S—, ═S, N(R′), NYR′, C(R′)(R″), CR′R″, C(O), C(O)O, C(O)OR′ or C(O)R′R″. More preferably, A is —O—, ═O, —S—, ═S, N(R′), NYR′, C(R′)(R″), CR′R″, C(O), C(O)O, C(O)OR′ or C(O)R′R″ and B is H.

More preferably, B is —O—, ═O, —S—, ═S, N(R′), NYR′, C(R′)(R″), CR′R″, C(O), C(O)O, C(O)OR′ or C(O)R′R″ and A is H.

Even more preferably, A is —O—, C(O), or C(O)O and B is H.

Even more preferably, B is —O—, C(O), or C(O)O and A is H.

W is a Linker well known in the art that is suitable for binding a targeting agent or vector to a small entity.

Preferably, W is selected but not limited to NR′, —O—, C(R′R″), branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, aminoacid, CO(CH₂)ₙ, [O(CH₂)ₙ—O(CH₂)ₙ]ₘ, or —O(CH₂)ₙ, [O(CH₂)ₙ—O(CH₂)ₙ]ₘ,

n=1 to 6 and m=1 to 6.

The targeting agent or vector is typically selected from the group consisting of a synthetic small molecule, a pharmaceutically active compound (i.e., a drug molecule), a metabolite, a signaling molecule, an hormone, a peptide, a protein, a receptor antagonist, a receptor agonist, a receptor inverse agonist, a vitamin, an essential nutrient, an amino acid, a fatty acid, a lipid, a nucleic acid, a mono-, di-, tri- or polysaccharide, a steroid, and the like. It will be understood that some of the aforementioned options will overlap in their meaning, i.e., a peptide may for example also be a pharmaceutically active compound, or a hormone may be a signaling molecule or a peptide hormone. Furthermore, it will be understood that also derivatives of the aforementioned substance classes are encompassed.

The targeting agent or vector (or, optionally, any metabolite), is preferably a moiety that specifically binds to a target site in a mammalian body. Specific binding in this context means that the compound targeting agent or vector for that matter, accumulates to a larger extent at this target site compared to the surrounding tissues or cells. For example, the targeting agent or vector may specifically bind to a receptor or integrin or enzyme that is preferentially expressed at a pathologic site within the mammalian body, or the targeting agent or vector may be specifically transported by a transporter that is preferentially expressed at a pathologic site within the mammalian body. In some embodiments, the receptor, integrin, enzyme, or transporter is exclusively expressed at a pathologic site within the mammalian body, i.e., to sites that are different or absent in healthy subjects, or vice versa. In this context, it will be understood that the targeting agent or vector preferably binds specifically to a receptor/or integrin/or enzyme/or transporter that is exclusively expressed or present at a pathologic site within the mammalian body and not expressed or present at a non-pathologic site, although the latter is—while no doubt highly desirable—rarely achieved in practice.

Examples for specific binding include, but are not limited to, specific binding to a site of infection, inflammation, cancer, platelet aggregation, angiogenesis, necrosis, ischemia, tissue hypoxia, angiogenic vessels, Alzheimer's disease plaques, atherosclerotic plaques, pancreatic islet cells, thrombi, serotonin transporters, neuroepinephrin transporters, LAT 1 transporters, apoptotic cells, macrophages, neutrophils, EDB fibronectin, receptor tyrosine kinases, cardiac sympathetic neurons, and the like.

In preferred embodiments, targeting agent or vector may be selected from the group consisting of a synthetic small molecule, a pharmaceutically active compound (drug), a peptide, a metabolite, a signaling molecule, a hormone, a protein, a receptor antagonist, a receptor agonist, a receptor inverse agonist, a vitamin, an essential nutrient, an amino acid, a fatty acid, a lipid, a nucleic acid, a mono-, di-, tri-, or polysaccharide, a steroid, a hormone and the like. More specifically, the targeting agent or vector may be selected from the group consisting of glucose, galactose, fructose, mannitol, sucrose, or stachyose and derivatives thereof, glutamine, glutamate, tyrosine, leucine, methionine, tryptophan, acetate, choline, thymidine, folate, methotrexate, Arg-Gly-Asp (RGD) peptides, chemotactic peptides, alpha melanotropin peptide, somatostatin, bombesin, human pro-insulin connecting peptides and analogues thereof, GPIIb/IIIa-binding compounds, PF4-binding compounds, αvβ3, αvβ6, or α4β1 integrin-binding compounds, somatostatin receptor binding compounds, GLP-1 receptor binding compounds, sigma 2 receptor binding compounds, sigma 1 receptor binding compounds, peripheral benzodiazepine receptor binding compounds, PSMA binding compounds, estrogen receptor binding compounds, androgen receptor binding compounds, serotonin transporter binding compounds, neuroepinephrine transporter binding compounds, dopamine transporter binding compounds, LAT1 transporter binding compounds and hormones such as peptide hormones, and the like.

In a preferred embodiment, the compound of Formula I is a compound of Formula I wherein E=H, OR′, SR′, NR′, or CR′ₚ and F=H, OR′, SR′, NR′, or CR′ₚ named compound of Formula I*.

Preferably, E=absent, H, C(R′)(R″), CR′R″, or W-Z, wherein W is a linker and Z is a targeting agent or vector. More preferably, E=H, C(R′)(R″), CR′R″, or W-Z, wherein W is a linker and Z is a targeting agent or vector.

Preferably, E=absent, H, C(R′)(R″), or CR′R″.

Preferably, F=absent, H, C(R′)(R″), CR′R″, or W-Z, wherein W is a linker and Z is a targeting agent or vector. More preferably, F=H, C(R′)(R″), CR′R″, or W-Z, wherein W is a linker and Z is a targeting agent or vector.

Preferably, F=absent, H, C(R′)(R″), or CR′R″.

Preferably, R′=H, OH, NH, branched or linear C₁-C₆ alkyl, branched or linear O—C₁-C₆ alkyl, branched or linear C₁-C₆ alkoxy, substituted or unsubstituted aryl, preferably phenyl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,

wherein n=1 to 6 and m=1 to 6. Preferably, n=1 to 3 or 4 to 6 and m=1 to 3 or 4 to 6.

Preferably, branched or linear C₁-C₆ alky is methyl, ethyl or butyl.

More preferably, R′=H, OH, methyl, ethyl or butyl.

Preferably, R″=H, OH, NH, branched or linear C₁-C₆ alkyl, branched or linear O—C₁-C₆ alkyl, branched or linear C₁-C₆ alkoxy, substituted or unsubstituted aryl, preferably phenyl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,

wherein n=1 to 6 and m=1 to 6. Preferably, n=1 to 3 or 4 to 6 and m=1 to 3 or 4 to 6. Preferably, branched or linear C₁-C₆ alky is methyl, ethyl or butyl.

More preferably, R″=H, OH, methyl, ethyl, butyl or phenyl.

Preferably, X=(CH₂)_{q} wherein q=1 or 2, preferably 1.

Suitable radioisotopes are well known in the art (Handbook of Nuclear Chemistry, Vol. 4 (Vol. Ed. F. Rösch; Ed. Vértes, A., Nagy, S., Klencsár, Z.) Kluver Academic Publishers, 2003; pp 119-202). The radioisotope is selected from the groups of ¹⁸F, ¹¹C, ¹²³I, ¹²⁴I, ¹²⁵I, ¹³¹I, ⁶⁴Cu²⁺, ⁶⁷Cu²⁺, ⁸⁹Zr, ⁶⁸Ga³⁺, ⁶⁷Ga³⁺, ¹¹¹In³⁺, ¹⁴C, ³H, ³²P, ⁸⁹Zr and ³³P.

In particular, for positron emission tomography (PET), ¹⁸F, ¹²³I, ¹²⁴I, ¹²⁵I, or ¹³¹I, are preferred as positron emitting radioisotopes, more preferably ¹⁸F.

The invention includes also all radioisotope counterpart i.e. cold isotope e.g ¹⁹F.

Preferably, when D is a radioisotope then C is H.

Preferably, when C is a radioisotope then D is H.

Preferably, E=H, OR′, SR′, NR′, or CR′ₚ and F=H, OR′, SR′, NR′, or CR′ₚ.

In a preferred embodiment, the compound of Formula II is a compound of Formula II wherein E=H, OR′, SR′, NR′, or CR′ₚ and F=H, OR′, SR′, NR′, or CR′ₚ named compound of Formula II*.

In a first embodiment., the invention relates to novel compounds of Formula II that are obtained from direct or indirect labeling with a radioisotope

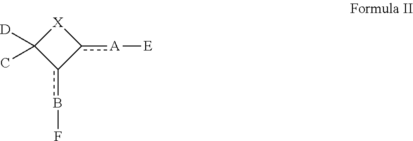

wherein

### A=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### B=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, —O— or S,

C=H, radioisotope, halogen or R′,
D=H, radioisotope, halogen or R′,
E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein
W is a linker and Z is a targeting agent,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′, or W-Z, wherein
W is a linker and Z is a targeting agent,
p=1 tO 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2,
with the proviso that at least C or D is radioisotope,
with the proviso that when A or B is ═O then E or F is absent and
with the proviso that at least E or F is W-Z
and pharmaceutical salt, diastereomere and enantiomere thereof.

In a second embodiment, the invention relates to novel compounds of Formula II that are labeled with a radioisotope wherein the compounds are suitable for indirect labeling.

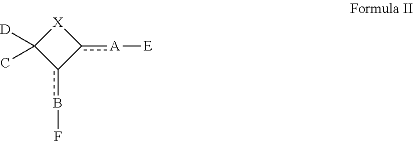

wherein

### A=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### B=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, —O— or S,

### Y=N, NR′, O or S,

C=H, radioisotope, halogen or R′,
D=H, radioisotope, halogen or R′,
E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′, C(O)R′R″, SO, SO2, SO2NR′,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2,
with the proviso that at least C or D is radioisotope,
with the proviso that when A or B is ═O then E or F is absent and
and pharmaceutical salt, diastereomere and enantiomere thereof.

Preferably, A-E and/or B-F are suitable moiety for coupling compounds of Formula II of the second embodiment to W-Z, wherein W is a linker and Z is a targeting agent or vector and correspond to compounds of Formula IIIa or IIIb.

Compounds of formula IIIa or IIIb are defined by the formula below

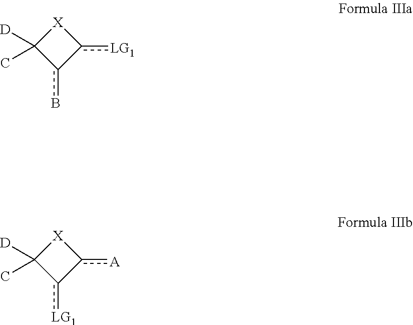

wherein

### LG₁=Leaving Group,

### A=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### B=H, —O—, ═O, —S—, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,

### Y=N, NR′, —O— or S,

C=H, radioisotope, halogen or R′,
D=H, radioisotope, halogen or R′,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl, substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,

### X=(CH₂)_{q} or C(R′R″),

q=0 to 2
and pharmaceutical salt, diastereomere and enantiomere thereof.

Preferably, LG₁ is A-E or B-F suitable for coupling compounds of Formula IIIa and IIIb with W-Z. Additionally, LG₁ is any coupling moieties known in the art suitable for coupling compounds of Formula IIIa and IIIb with W-Z wherein the obtained compounds are compounds of Formula II of the first embodiment with A-W-Z and/or B-W-Z or compounds of Formula II of the first embodiment with A and/or B are a bond.

In a third embodiment, the invention relates to novel compounds of Formula II, IIIa or IIIb wherein

A=bond, —O—, —S—, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,
B=bond, —O—, —S—, N, N(R′), NYR′, P(R′)(R″), P(O)(R′)R″, C(R′)(R″), CR′R″, C(O), C(O)O, C(O)OR′, C(O)R′R″, SO, SO2, or SO2NR′.

Preferably, A and/or B is bond.

Embodiments and preferred features can be combined together and are within the scope of the invention.

Invention compounds are but not limited to

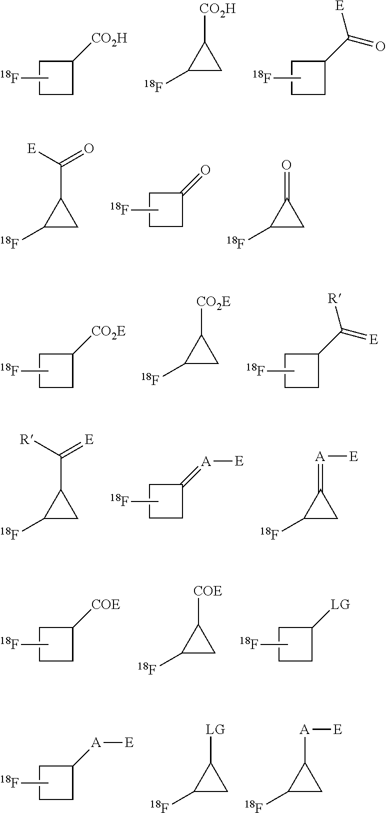

A, E and R′ as disclosed above

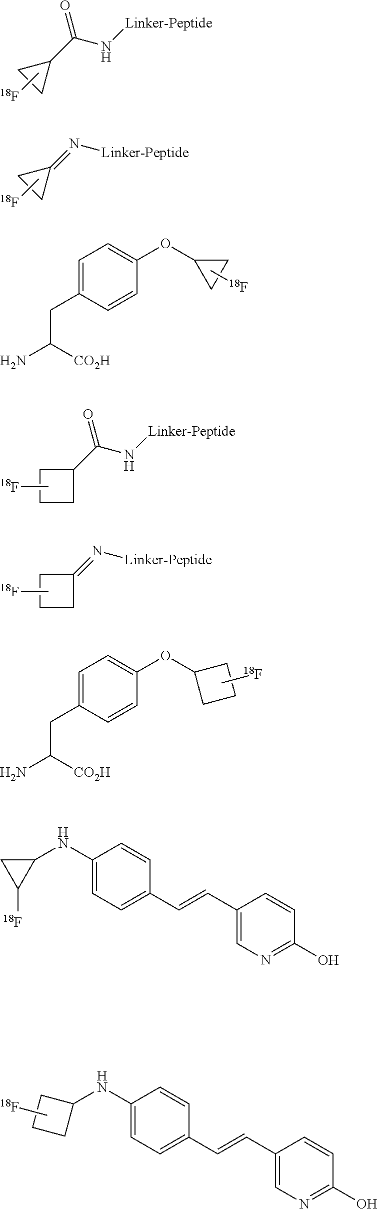

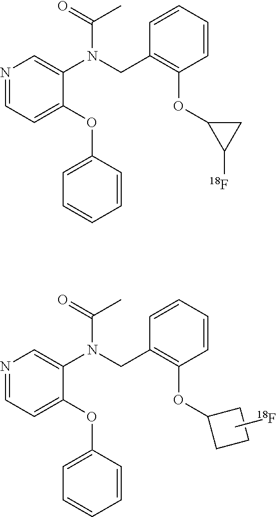

### trans-Methyl 3-fluorocyclobutanecarboxylate

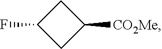

### trans-Benzyl 3-fluorocyclobutanecarboxylate

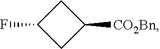

### N-(2-{4-[4-(Benzyloxy)phenyl]piperazin-1-yl}-2-oxoethyl)-3-[cis-(3-fluorocyclobutyl)oxy]benzamide

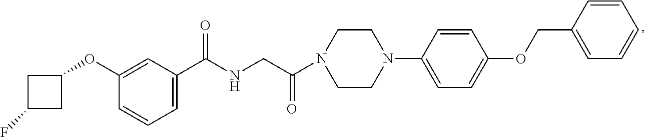

### O-(cis-3-[¹⁸F]-Fluorocyclobutyl)-L-tyrosine

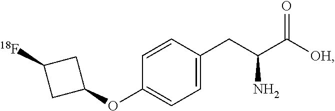

### N-(2-{4-[4-(Benzyloxy)phenyl]piperazin-1-yl}-2-oxoethyl)-3-[cis-(3-[¹⁸F]-fluorocyclobutyl)oxy]benzamide

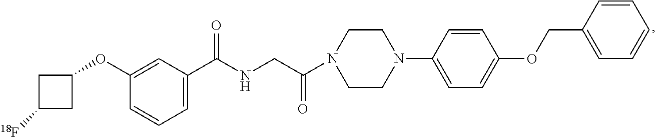

### trans-3-[¹⁸F]Fluorocyclobutyl toluene-4-sulfonate

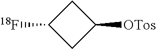

In a third aspect the invention relates to methods of preparing compound of Formula I or II, see Scheme 8.

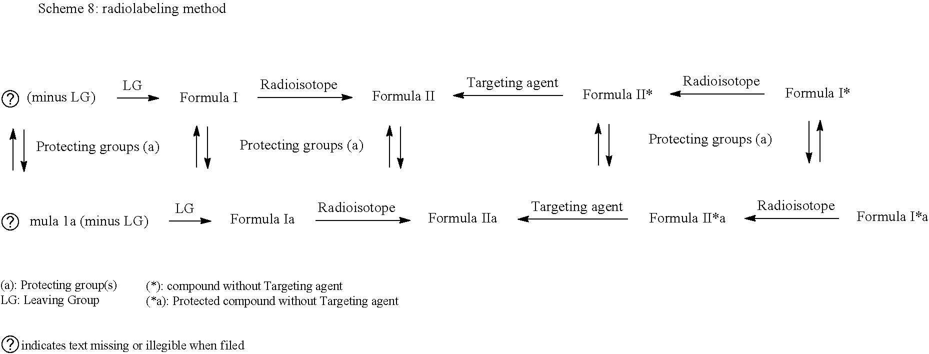

In one embodiment, the method for obtaining compound of Formula I comprising the steps
   Optionally adding protecting group(s) to a compound of Formula I having no leaving group (Formula I (minus LG)) for obtaining a compound of Formula Ia (minus LG),
   Reacting the compound compound of Formula I having no leaving group (Formula I (minus LG)) with LG for obtaining a compound of Formula I or Ia, and
   Optionally unprotecting compound of Formula Ia for obtaining a compound of Formula I.

Preferably, the method for obtaining compound of Formula I comprises the step of
Reacting the compound of Formula I having no leaving group (Formula I (minus LG)) with LG for obtaining a compound of Formula I.

The compounds of Formula Ia (minus LG) and Ia have their functional group(s) protected with a suitable protecting group(s),
In second embodiment, the method is a direct labeling method for obtaining compound of Formula II comprising the steps
   Optionally adding protecting group(s) to a compound of Formula I for obtaining a compound of Formula Ia,
   Radiolabeling of compound of Formula I or Ia with a radioisotope for obtaining a compound of Formula II or IIa, and
   Optionally unprotecting compound of Formula IIa for obtaining a compound of Formula II.

Preferably, the method for obtaining compound of Formula II comprises the step of
Radiolabeling of compound of Formula I with radioisotope for obtaining a compound of Formula II,
In third embodiment, the method is a indirect labeling method for obtaining compound of Formula II comprising the steps
   Optionally adding protecting group(s) to a compound of Formula I* for obtaining a compound of Formula I*a,
   Radiolabeling of compound of Formula I* or I*a (compound of Formula I without targeting agent or vector moiety) with a radioisotope for obtaining a compound of Formula II* or II*a (compound of Formula II without targeting agent or vector moiety),
   Reacting a compound of Formula II* or II*a (compound of Formula II without targeting agent or vector moiety) with a targeting agent or vector moiety) for obtaining a compound of Formula II or IIa, and
   Optionally unprotecting compound of Formula IIa for obtaining a compound of Formula II.
Preferably, the method for obtaining compound of Formula II comprises the steps of
   Radiolabeling of compound of Formula I* (compound of Formula I without targeting agent or vector moiety) with radioisotope for obtaining a compound of Formula II* (compound of Formula II without targeting agent or vector moiety), and
   Reacting a compound of Formula II* (compound of Formula II without targeting agent or vector moiety) with a targeting agent or vector moiety) for obtaining a compound of Formula II.
More preferably, the method for obtaining compound of Formula II comprises the step of
   Radiolabeling of compound of Formula I wherein E=absent, H, OR′, SR′, NR′, CR′ₚ and F=absent, H, OR′, SR′, NR′, CR′ₚ, p=1 to 3 with radioisotope for obtaining a compound of Formula II wherein E=absent, H, OR′, SR′, NR′, CR′ₚ and F=absent, H, OR′, SR′, NR′, CR′ₚ, p=1 to 3.

Embodiments and preferred features of compounds of Formula I, II, IIIa and IIIb are enclosed herein.

In a fourth aspect the invention relates to pharmaceutical compositions comprising compounds of Formula I, Ia, I*, I*a or II, IIa, II*, II*a and pharmaceutically acceptable salt of an inorganic or organic acid thereof, a hydrate, a complex, an ester, an amide, a solvate or a prodrug thereof and a pharmaceutical acceptable carrier, diluent, excipient or adjuvant.

In one embodiment, the pharmaceutical compositions comprise a compound of Formula I that is a pharmaceutical acceptable salt, hydrate, complex, ester, amide, solvate or a prodrug thereof.

In a fifth aspect the invention relates to a kit for preparing a radiopharmaceutical composition, said kit comprising a sealed vial containing a predetermined quantity of the compound of Formula I or II, and a pharmaceutically acceptable salts of inorganic or organic acids thereof, hydrates, complexes, esters, amides, solvates and prodrugs thereof and further optionally an acceptable carrier, diluent, excipient or adjuvant supplied as a mixture with the compound having general chemical Formula I or II. More preferably, the present invention relates to a kit comprising a compound or composition, as defined herein above, in powder form, and a container containing an appropriate solvent for preparing a solution of the compound or composition for administration to an animal, including a human.

In a sixth aspect the invention relates to compound of Formula II wherein the compound is deprotected or unprotected for imaging by positron emission tomography (PET) or single-photon emission computed tomography (SPECT).

The invention relates to the use of compound of Formula II wherein the compound is deprotected or non-deprotected for the manufacture of radiopharmaceutical for positron emission tomography (PET) or single-photon emission computed tomography (SPECT) imaging.

Various diseases and physiological disfunctionment can been identify depending on the targeting agent.

### DEFINITION

For the purposes of the present invention, the term “targeting agent” or vector shall have the following meaning: The targeting agent or vector is a compound or moiety that targets or directs the radionuclide attached to it to a specific site in a biological system. A targeting agent or vector can be any compound or chemical entity that binds to or accumulates at a target site in a mammalian body, i.e., the compound localizes to a greater extent at the target site than to surrounding tissue.

The term “alkyl” as used herein refers to C₁ to C₆ straight or branched alkyl groups, e.g., methyl, ethyl, propyl, isopropyl, n-butyl, t-butyl, n-pentyl, or neopentyl. Alkyl groups can be perfluorated or substituted by one to five substituents selected from the group consisting of halogen, hydroxyl, C₁-C₄ alkoxy, or C₆-C₁₂ aryl (which can be substituted by one to three halogen atoms). More preferably, alkyl is a C₁ to C₄ or C₁ to C₃ alkyl.

The term “alkenyl” as used herein refers to a straight or branched chain monovalent or divalent radical, containing at least one double bond and having from two to ten carbon atoms, e.g., ethenyl, prop-2-en-1-yl, but-1-enyl, pent-1-enyl, penta-1,4-dienyl, and the like.

The term “alkynyl” as used herein refers to a substituted or unsubstituted straight or branched chain monovalent or divalent radical, containing at least one triple bond and having from two to ten carbon atoms, e.g., ethynyl, prop-1-ynyl, but-1-ynyl, pent-1-ynyl, pent-3-ynyl, and the like.

Alkenyl and alkynyl groups can be substituted by one or more substituents selected from the group consisting of halogen, hydroxyl, alkoxy, —CO₂H, —CO₂Alkyl, —NH₂, —NO₂, —N₃, —CN, C₁-C₂₀ acyl, or C₁-C₆ acyloxy.

The term “aryl” as used herein refers to an aromatic carbocyclic or heterocyclic moiety containing five to 10 ring atoms, e.g., phenyl, naphthyl, furyl, thienyl, pyridyl, pyrazolyl, pyrimidinyl, oxazolyl, pyridazinyl, pyrazinyl, chinolyl, or thiazolyl. Aryl groups can be substituted by one or more substituents selected from the group consisting of halogen, hydroxyl, alkoxy, —CO₂H, —CO₂Alkyl, —NH₂, Alkyl-NH₂, C₁-C₂₀ alkyl-thiolanyl, —NO₂, —N₃, —CN, C₁-C₂₀ alkyl, C₁-C₂₀ acyl, or C₁-C₂₀ acyloxy. The heteroatoms can be oxidized, if this does not cause a loss of aromatic character, e.g., a pyridine moiety can be oxidized to give a pyridine N-oxide.

Whenever the term “substituted” is used, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a pharmaceutical composition. The substituent groups may be selected from halogen atoms, hydroxyl groups, nitro, (C₁-C₆)carbonyl, cyano, nitrile, trifluoromethyl, (C₁-C₆)sulfonyl, (C₁-C₆)alkyl, (C₁-C₆)alkoxy and (C₁-C₆)sulfanyl.

Halogen means Chloro, Iodo, Fluoro and bromo. Preferably, halogen means iodo or fluoro.

Radioisotope of the invention are PET radioisotopes and SPECT radioisotopes. Suitable PET radioisotopes (29) are well known in the art (Handbook of Nuclear Chemistry, Vol. 4 (Vol. Ed. F. Rösch; Ed. Vértes, A., Nagy, S., Klencsár, Z.) Kluver Academic Publishers, 2003; pp 119-202). Suitable radioisotope-contained complexes for SPECT imaging (30) are well known in the art ((Handbook of Nuclear Chemistry, Vol. 4 (Vol. Ed. F. Rösch; Ed. Vértes, A., Nagy, S., Klencsár, Z.) Kluver Academic Publishers, 2003; pp 279-310). The radioactive label is a radioisotope-contained complex and/or is a moiety or atom that is covalently bond to the compound or complex. The radioisotope is selected from the groups of ^{99m}Tc, ¹⁸F, ¹¹C, ¹²³I, ¹²⁴I, ¹²⁵I, ¹³¹I, ⁶⁴Cu²⁺, ⁶⁷Cu²⁺, ⁸⁹Zr, ⁶⁸Ga³⁺, ⁶⁷Ga³⁺, ¹¹¹In³⁺, ¹⁴C, ³H, ³²P, ⁸⁹Zr and ³³P.

In particular, for positron emission tomography (PET), ¹⁸F, ⁶⁸Ga, ⁶⁴Cu or ¹²⁴I are preferred as positron emitting radioisotopes, more preferably ¹⁸F or ⁶⁸Ga. For single-photon emission computed tomography (SPECT), ¹²³I, ¹²⁵I, ¹¹¹In, and ^{99m}Tc are preferred, more preferably ¹²³I or ^{99m}Tc.

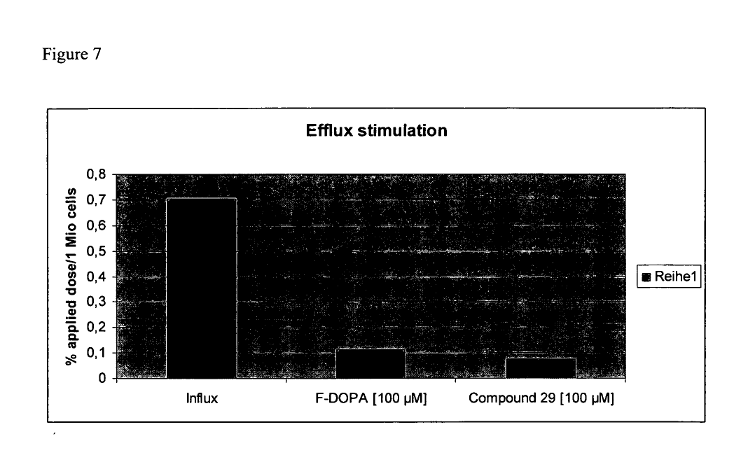

The term “leaving group” as employed herein by itself or as part of another group is known or obvious to someone skilled in the art, and means that an atom or group of atoms is detachable from a chemical substance by a nucleophilic agent. Examples are given e.g. in Synthesis (1982), p. 85-125, table 2 (p. 86; (the last entry of this table 2 needs to be corrected: “n-C₄F₉S(O)₂—O— nonaflat” instead of “n-C₄H₉S(O)₂—O— nonaflat”), Carey and Sundberg, Organische Synthese, (1995), page 279-281, table 5.8; or Netscher, Recent Res. Dev. Org. Chem., 2003, 7, 71-83, scheme 1, 2, 10 and 15 and others). (Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50, explicitly: scheme 4 pp. 25, scheme 5 pp 28, table 4 pp 30, FIG. 7 pp 33).

Whenever the term “aminoacid” is used, it is meant to indicate that

The term “N-protecting group” (amine-protecting group) as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, which is hereby incorporated herein by reference.

The term “O-protecting group” as employed herein refers to a carboxylic acid protecting group employed to block or protect the carboxylic acid functionality while the reactions involving other functional sites of the compound are carried out. Carboxy protecting groups are disclosed in Greene, “Protective Groups in Organic Synthesis” pp. 152-186 (1981), which is hereby incorporated herein by reference. Such carboxy protecting groups are well known to those skilled in the art, having been extensively used in the protection of carboxyl groups. Representative carboxy protecting groups are alkyl (e.g., methyl, ethyl or tertiary butyl and the like); arylalkyl, for example, phenethyl or benzyl and substituted derivatives thereof such as alkoxybenzyl or nitrobenzyl groups and the like.

The term “protein”, as used herein, means any protein, including, but not limited to peptides, enzymes, glycoproteins, hormones, receptors, antigens, antibodies, growth factors, etc., without limitation, having at least about 20 or more amino acids (both D and/or L forms thereof). Included in the meaning of protein are those having more than about 20 amino acids, more than about 50 amino acid residues, and sometimes even more than about 100 or 200 amino acid residues.

The term “peptide” as used herein refers to any entity comprising at least one peptide bond, and can comprise either D and/or L amino acids. The meaning of the term peptide may sometimes overlap with the term protein as defined herein above. Thus, peptides according to the present invention have at least 2 to about 100 amino acids, preferably 2 to about 50 amino acids. However, most preferably, the peptides have 2 to about 20 amino acids, and in some embodiments between 2 and about 15 amino acids.

The term “small molecule” is intended to include all molecules that are less than about 1000 atomic units. In certain embodiments of the present invention, the small molecule is a peptide which can be from a natural source, or be produced synthetically. In other embodiments, the small molecule is an organic, non-peptidic/proteinaceous molecule, and is preferably produced synthetically. In particular embodiments, the small molecule is a pharmaceutically active compound (i.e., a drug), or a prodrug thereof, a metabolite of a drug, or a product of a reaction associated with a natural biological process, e.g., enzymatic function or organ function in response to a stimulus. small molecule has generally a molecular weight of between about 75 to about 1000.

### EXPERIMENTAL PART

Abbreviations

| | |
|---|---|
| aq | aqueous |
| b.p. | boiling point |
| d | doublet |
| dd | doublet of quartet |
| h | hour |
| K₂₂₂ | 4,7,13,16,21,24-hexaoxa-1,10- |
| | diazabicyclo[8.8.8]-hexacosane |
| m | multiplet |
| min | minute |
| NMR | nuclear magnetic resonance |
| | spectroscopy: chemical shifts (δ) are |
| | given in ppm. |
| q | quartet |
| quint | quintet |
| r.t. | room temperature |
| RT | retention time |
| s | singulet |
| sat. | saturated |
| t | triplet |
| TLC | thin layer chromatography |

### 1. Experimental Chemistry

### 1.1 Cold synthesis of trans-3-fluorocyclobutanecarboxylic acid (5)—Synthesis path 1

Compound 5 is a prosthetic group and can be afterward coupled to a biological molecule such as peptide or small molecule by known coupling methods.

### Methyl 3-oxocyclobutanecarboxylate (1)

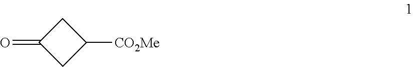

A mixture of 3-oxocyclobutanecarboxylic acid (50 g, 438 mmol), methanol (17.75 mL; 438 mmol), 4-N,N-dimethylaminopyridine (5.37 g, 43.7 mmol) and N-[3-(dimethylamino)propyl]-N′-ethylcarbodiimide hydrochloride (126 g, 657 mmol) in dichloromethane (2500 mL) was stirred overnight at room temperature. The mixture was washed with water (3*200 mL) and the combined aqueous phase was back extracted with dichloromethane (2*100 mL). The combined organic phase was washed with 0.5M hydrochloric acid (200 mL), half saturated sodium hydrogen carbonate (100 mL), water (100 mL) and brine (100 mL). The mixture was dried over sodium sulfate and concentrated to dryness in vacuo to afford methyl 3-oxocyclobutanecarboxylate (1) (54 g; 421 mmol; 96%), which was used without further purification.

### cis-Methyl 3-hydroxycyclobutanecarboxylate (2)

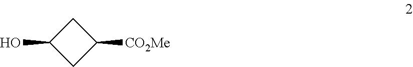

A solution of methyl 3-oxocyclobutanecarboxylate (1) (50 g, 390 mmol) in methanol was cooled on an ice bath. After the portion wise addition of sodium borohydride (15 g, 397 mmol) the mixture was stirred at 0° C. for 2 hrs by which time TLC-analysis (dichloromethane/10% methanol, potassium permanganate) showed completion of the reaction. After the addition of 4M hydrochloric acid in dioxane until a pH of 7 was reached the mixture was diluted with methanol (1000 mL) and stirred overnight at room temperature. The mixture was evaporated to dryness and re-suspended in dichloromethane₂ (300 mL). This was washed with water (2*150 mL), sodium hydrogen carbonate-sat. (2*150 mL), water (150 mL) and brine (100 mL). Solvents were removed under reduced pressure and the crude compound was purified by column chromatography (ethyl acetate/heptane=1:1) to give (cis)-methyl 3-hydroxycyclobutanecarboxylate (2) (20.06 g; 154 mmol, 39%), predominantly cis. Compound 2 is the precursor for cold [¹⁹F]-labeling wherein Hydroxy is replaced by [¹⁹F].

### cis-Methyl 3-(tosyloxy)cyclobutanecarboxylate (3)

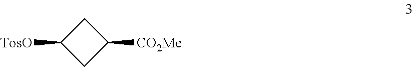

To a solution of (cis)-methyl 3-hydroxycyclobutanecarboxylate (2) (10 g, 76.8 mmol) in dichloromethane (300 mL) was added pyridine (9.4 mL) and tosyl anhydride (27.56 g, 84.5 mmol). The mixture was stirred overnight at room temperature. The mixture was concentrated in vacuo, resuspended in diethyl ether (200 mL) and washed with 0.5M hydrochloric acid (2*60 mL), sodium hydrogen carbonate-sat (2*60 mL), water (60 mL) and brine (50 mL), and then dried over sodium sulfate, filtered and concentrated to yield the title compound as an oil, predominantly cis (18 g). This was purified by column chromatography (ethyl acetate/heptane=1:4) to afford a fraction of predominantly cis-Methyl 3-(tosyloxy)cyclobutanecarboxylate (3) (14.9 g) and a contaminated fraction (1.5 g; cis-Methyl 3-(tosyloxy)cyclobutanecarboxylate=1:1). The first fraction (14.9 g) was further purified by column chromatography (silica gel, 1200 ml); and a gradient ethyl acetate/heptane=0:1 to 1:4 as an eluent to give a pure fraction of cis-Methyl 3-(tosyloxy)cyclobutanecarboxylate (3) (4.9 g), a fraction contaminated with the trans-isomer (4.95 g) and two fractions more contaminated (0.84 resp. 1.38 g). Cy=45-55%.

Compound 3 is the precursor for hot [¹⁸F]-labeling wherein tosylate is replaced by [¹⁸F].

### trans-Methyl 3-fluorocyclobutanecarboxylate (4)

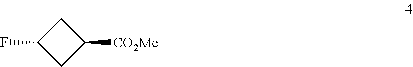

trans-Methyl 3-fluorocyclobutanecarboxylate (4) is obtained from cis-Methyl 3-hydroxycyclobutanecarboxylate (2) with the same method as described for trans-Benzyl 3-fluorocyclobutanecarboxylate (9).

### trans-3-Fluorocyclobutanecarboxylic acid (5)

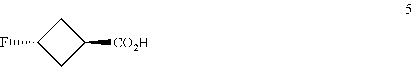

### trans-3-Fluorocyclobutanecarboxylic acid (5) is obtained from trans-Methyl 3-fluorocyclobutanecarboxylate (4) with the same method as described below

### 1.2 Cold synthesis of trans-3-fluorocyclobutanecarboxylic acid (5)—Synthesis path 2

### Benzyl 3-oxocyclobutanecarboxylate (6)

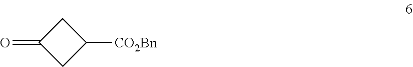

To 3-oxocyclobutanecarboxylic acid (10 g, 87.6 mmol) in dry toluene (100 mL) was added benzyl alcohol (9.1 mL, 87.6 mmol) and p-toluenesulphonic acid (0.4 g, 2.1 mmol). The reaction was heated under Dean-Stark conditions for 3 h. The reaction was concentrated to dryness in vacuo to afford the crude product. Purification using silica chromatography (ethyl acetate/hexane, 0-100% gradient) gave benzyl 3-oxocyclobutanecarboxylate (6) (16 g; 89%) as a colourless oil.

¹H NMR CDCl₃: δ ppm 7.30 (s, 5H), 5.10 (s, 2H), 3.43-3.29 (m, 2H), 3.28-3.13 (m, 3H).

### cis-Benzyl 3-hydroxycyclobutanecarboxylate (7)

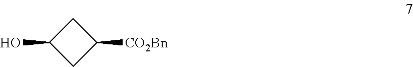

A solution of benzyl 3-oxocyclobutanecarboxylate (6) (16 g, 78.3 mmol) in dry tetrahydrofurane under Argon was cooled to −78° C. To the solution was added dropwise 1M lithium tri-tert-butoxyaluminohydride in tetrahydrofurane (78.4 mL, 78.3 mmol). After complete addition the reaction was stirred at −78° C. for 3 h. The reaction was quenched by the addition of sat. ammonium chloride (aq) (100 mL). The organics were extracted with ethyl acetate, dried over magnesium sulfate, filtered and the solvents were removed under reduced pressure. The crude compound was purified by column chromatography (ethyl acetate/hexane, 0-100% gradient) to give cis-benzyl 3-hydroxycyclobutanecarboxylate (7) (14.5 g; 90%), predominantly cis, as a colourless oil.

¹H NMR CDCl₃: δ ppm 7.30 (s, 5H), 5.08 (s, 2H), 4.19-4.01 (m, 1H), 2.65-2.47 (m, 3H), 2.22-2.06 (m, 2H)

Compound 7 is the precursor for cold [¹⁹F]-labeling wherein hydroxy is replaced by [¹⁹F].

### (cis)-Benzyl 3-(tosyloxy)-cyclobutanecarboxylate (8)

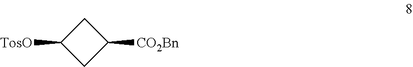

To a solution of cis-benzyl 3-hydroxycyclobutanecarboxylate (7) (2.24 g, 11 mmol) in dry dichloromethane (75 mL) was added pyridine (5.34 ml, 66 mmol). To this solution was added slowly dropwise a solution of p-toluenesulfonyl chloride (4.19 g, 22 mmol) in dry dichloromethane (23 mL). The mixture was stirred at room temperature for 72 h. The mixture was concentrated in vacuo and resuspended in dichloromethane (300 mL) and washed with 2M hydrochloric acid (150 mL), water (150 mL), 2M sodium hydroxide (150 mL) and water (150 mL). The organics were dried over sodium sulfate, filtered and concentrated to yield a yellow oil. This was purified by column chromatography (ethyl acetate/hexane, 0-100% gradient) to afford the cis-benzyl 3-(4-methylbenzenesulfonyl)-cyclobutanecarboxylate (8) (2.1 g, 53.6%) as white crystals.

¹H NMR CDCl₃: δ ppm 7.77 (d, 2H), 7.36-7.29 (m, 7H), 5.09 (s, 2H), 4.74 (quint, 1H), 2.73-2.61 (m, 1H), 2.54-2.37 (m, 4H), 2.45 (s, 3H)

¹³C NMR CDCl₃: δ ppm 172.91, 144.87, 135.54, 133.74, 129.82, 128.54, 128.30, 128.12, 127.74, 69.49, 66.66, 34.07, 29.57, 21.58

Cis isomer confirmed by ¹H NOESY indicating a clear Overhauser effect between protons at 4.74 ppm and 2.70 ppm (corresponding to the methine protons in the cyclobutyl ring)

Compound 8 is the precursor for hot [¹⁸F]-labeling wherein tosylate is replaced by [¹⁸F].

### trans-Benzyl 3-fluorocyclobutanecarboxylate (9)

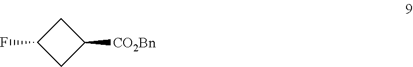

To a solution of cis-benzyl 3-hydroxycyclobutanecarboxylate (7) (6.4 g, 31 mmol) in dry dichloromethane (50 mL) and dry tetrahydrofurane (50 mL) was cooled to −78° C. To this solution was added dropwise Deoxo-Fluor® (2.33M in tetrahydrofurane, 20 mL, 46.6 mmol). Upon complete addition the yellow solution was stirred for 3 h at −78° C. The reaction mixture was allowed to warm to r.t. and stirred at r.t. for 50 min. The reaction was quenched by careful addition of 2M sodium hydroxide (50 mL, gas evolution). The organics were extracted with ethyl acetate, dried over sodium sulfate, filtered and concentrated in vacuo. The crude product was purified by triple distillation (b.p. 102-104° C. at 0.05 mbar) to afford trans-benzyl 3-fluorocyclobutanecarboxylate (9) (2.7 g, 42%) as a colourless oil.

¹H NMR CDCl₃: δ ppm 7.40-7.33 (m, 5H), 5.24 (dq, 1H), 5.14 (s, 2H), 3.22-3.11 (m, 1H), 2.69-2.41 (m, 4H)

¹³C NMR CDCl₃: δ ppm 175.04, 135.76, 128.58, 128.30, 128.11, 86.20, 207.47, 66.59, 34.10, 30.92

Trans isomer confirmed by ¹H NOESY indicating no Overhauser effect between protons at 5.24 ppm and 3.22-3.11 ppm (corresponding to the methine protons in the cyclobutyl ring)

### trans-3-Fluorocyclobutanecarboxylic acid (5)

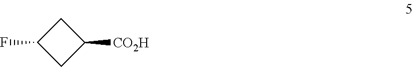

To a solution of trans-benzyl 3-fluorocyclobutanecarboxylate (9) (2.7 g, 13 mmol) in methanol (50 mL) was added to a slurry of Pd/C (10%, 200 mg) in methanol (50 mL) under Argon. The flask was evacuated and re-filled with H₂-gas. The reaction was stirred at r.t. for 5 h. TLC indicated no starting material. The reaction mixture was filtered through Celite and concentrated in vacuo. The crude product was purified by triple distillation (b.p. 83-85° C. at 0.9-1.0 mbar) to afford trans-3-fluorocyclobutanecarboxylic acid (5) (1.53 g, quantitative) as a crystalline white solid.

¹H NMR CDCl₃: δ ppm 5.23 (dq, 1H), 3.20-3.08 (m, 1H), 2.72-2.42 (m, 4H)

¹³C NMR CDCl₃: δ ppm 182.04, 86.0, 34.05, 30.85. Trans isomer confirmed by ¹H NOESY indicating no Overhauser effect between protons at 5.23 ppm and 3.20-3.08 ppm (corresponding to the methine protons in the cyclobutyl ring)

### 1.3 Cold synthesis of trans-3-fluorocyclobutanol (15) and trans-3-fluorocyclobutyl 4-methylbenzenesulfonate (16) and precursors for [¹⁸F] labeling (12,13) scheme 10

Compound 15 is a prosthetic group and compound 16 is prosthetic group substituted with a leaving group suitable for coupling with amino acid or peptide, scheme 9.

### cis-3-(Benzyloxy)cyclobutan-1-ol (10)

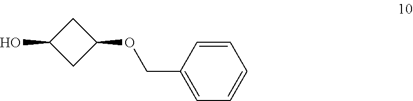

To an ice-cooled solution of 3-(benzyloxy)cyclobutanone (Chem. Ber., 1957, 90, 1424 and Appl. Radiat. Isot. 2003, 58, 657, 11.16 g; 63.3 mmol) in ethanol (170 mL) was added sodium borohydride (2.4 g; 63.4 mmol) in portions (only the first portion showed an exotherm). The mixture was stirred at 0° for 3 h by which time TLC-analysis (ethyl acetate/heptane=1:2) showed complete conversion of starting material. The mixture was filtered through Celite and evaporated to dryness. ¹H-NMR showed that boronic salts were isolated. The mixture was re-dissolved in methanol (250 mL) with gas evolution. To the solution was added 1M hydrochloric acid (about 15 mL) in 1 mL portions until no more change in pH (about 7) was observed. The mixture was concentrated in vacuo and stripped with ethanol. The mixture was partitioned between water (30 mL) and diethyl ether (60 mL) Phases were separated and the aqueous phase was extracted with diethyl ether (2*60 mL). The combined organic phase was washed with 1M sodium carbonate, water and brine and dried over sodium sulfate.

Concentration to dryness under reduced pressure gave cis-3-(benzyloxy)cyclobutan-1-ol (10) (10.33 g; 57.9 mmol; 91.5%) as predominantly cis. Compound 10 is the precursor for cold [¹⁹F]-labeling wherein hydroxy is replaced by [¹⁹F].

### cis-3-Benzyloxycyclobutyl toluene-4-sulfonate (11)

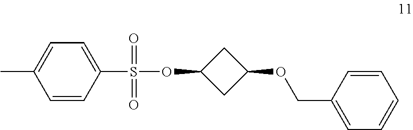

A solution of cis-3-(benzyloxy)cyclobutan-1-ol (10) (11.3 g; 63.4 mmol) in dichloromethane (280 mL) was cooled to 0° C. and triethyamine (13.2 mL) was added followed by the dropwise addition of p-toluenesulfonyl chloride (14.5 g; 76 mmol) in dichloromethane (40 mL). The mixture was stirred at 0° C. for 3 h and an additional 40 h at room temperature. The mixture was washed with water (2*50 mL) and the aqueous phase was back extracted with dichloromethane (50 mL). The combined organic phase was washed with brine and dried over sodium sulfate and concentrated in vacuo to give crude 6 (22.59 g) as predominantly cis-3-benzyloxycyclobutyl toluene-4-sulfonate. Purification by column chromatography (silica gel (600 g); ethyl acetate/heptane (1:6) as an eluent afforded a pure fraction of cis-3-benzyloxycyclobutyl toluene-4-sulfonate (6.08 g;) and an impure fraction (7.8 g) which was purified a second time to afford pure cis-3-benzyloxycyclobutyl toluene-4-sulfonate (6.1 g) and a contaminated fraction (2.7 g) as well as some starting material (1.77 g). Total yield of cis-3-benzyloxycyclobutyl toluene-4-sulfonate (11) (12.1 g; 36.4 mmol; 57.5%). The compound also crystallizes from ethyl acetate/heptane.

### (cis)-3-Hydroxycyclobutyl toluene-4-sulfonate (12)

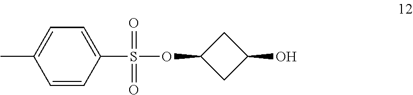

cis-3-(Benzyloxy)cyclobutyl toluene-4-sulfonate (11) (6.1 g, 18.35 mmol, after second column chromatography of fraction 2) was dissolved in ethanol (110 mL) and nitrogen was bubbled through the solution. After the addition of Pd-charcoal (10%; 2.28 g) the mixture was hydrogenated under balloon pressure overnight. The catalyst was removed by filtration over Celite. Evaporation of all volatiles in vacuo afforded the title compound as an oil (4.1 g; 16.9 mmol; 92%).

### cis-Cyclobutane-1,3-diyl bis(toluene-4-sulfonate (13)

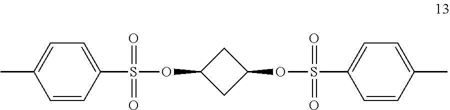

A solution of cis-3-hydroxycyclobutyl toluene-4-sulfonate (12) (4.29 g; 17.7 mmol) in dichloromethane was cooled to 0° C. Pyridine (2.9 mL) was added, followed by the addition of p-toluenesulphonic anhydride (8.67 g; 26.6 mmol; 1.5 equiv.). The mixture was stirred over the weekend at room temperature. The mixture was concentrated to dryness and re-suspended in diethyl ether (750 mL). The suspension was washed with 0.5M hydrochloric acid (2*10 mL), sodium hydrogen carbonate-sat. (15 mL) and brine. The mixture was dried over sodium sulfate and evaporated to dryness under reduced pressure to give crude cis-Cyclobutane-1,3-diyl bis(toluene-4-sulfonate) (5.3 g).

A second batch of crude cis-Cyclobutane-1,3-diyl bis(toluene-4-sulfonate) (525 mg) was prepared from cis-3-hydroxycyclobutyl toluene-4-sulfonate (0.9 g). The crude batches were combined and purified by column chromatography with ethyl acetate/heptane (1:6) as an eluent to give pure cis-Cyclobutane-1,3-diyl bis(toluene-4-sulfonate) (4.95 g; 12.5 mmol) and a second pure fraction (400 mg; 1 mmol). Total yield of cis-Cyclobutane-1,3-diyl bis(toluene-4-sulfonate) (5.35 g; 13.5 mmol; 64%).

Compound 13 is the precursor for hot [¹⁸F]-labeling wherein one Tosylate is replaced by [¹⁸F].

### trans-(3-Fluorocyclobutyl)benzyl ether (14)

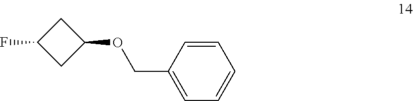

To an ice-cooled solution of 0.9 g (5.54 mmol) cis-3-(benzyloxy)cyclobutan-1-ol (10) in 25 mL dry dichloromethane 0.86 ml (6.54 mmol) diethylaminosulfur trifluoride was added under nitrogen. The mixture was stirred for 2 h at 0° C. and then at 25° C. overnight. The yellow-brown reaction mixture was washed with 20 mL water, the organic phase was separated and the aqueous phase was extracted (2× dichloromethane). The organic layers were combined, dried over sodium sulfate, filtered and concentrated in vacuo. The residue purified by silica chromatography with a gradient of ethyl acetate and hexane. Product showed a single spot in TLC (ethyl acetate/hexane 1:2, R_{f}˜0.66).

Yield: 306 mg (30%)

¹H NMR (400 MHz, CDCl₃): δ ppm 2.34-2.63 (m, 4H) 4.31-4.41 (m, 1H) 4.43 (s, 2H) 5.14-5.40 (dm, 1H) 7.28-7.58 (m, 5H)

¹⁹F-NMR (400 MHz, CDCl₃): δ ppm=−176.44

### trans-3-Fluorocyclobutan-1-ol (15)

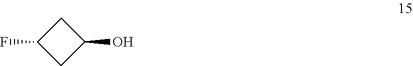

A solution of 152 mg (0.84 mmol) of trans-(3-Fluorocyclobutyl)benzyl ether (14) in 10 mL methanol was stirred with 140 mg 10% palladium on charcoal (50% wet). The mixture was stirred under a positive pressure of hydrogen at 25° C. The mixture was filtered and the solvent evaporated. Product showed a single spot in TLC (ethyl acetate/hexane 1:2, R_{f}˜0.26)

Yield: 54 mg (71%)

¹H NMR (400 MHz, CDCl₃): δ ppm 2.23-2.62 (dm, 4H) 4.64-4.68 (m, 1H) 5.17-5.37 (dm, 2H)

¹⁹F NMR (376 MHz, CDCl₃): δ ppm −178.28 (m, 1F)

### trans-3-Fluorocyclobutyl toluene-4-sulfonate (16)

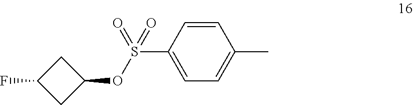

A solution of 50 mg (0.56 mmol) trans-3-fluorocyclobutan-1-ol (15) in 5 ml dichloromethane was cooled to 0° C. and 82 μL (1 mmol) pyridine was added followed by 201 mg (0.62 mmol) of p-toluenesulfonic anhydride. The mixture was stirred for 5 h at 0° C. under nitrogen atmosphere and let reach 25° C. overnight. The yellow solution was concentrated in vacuo. The resulting residue was dissolved in 5 mL hydrochloric acid (0.5 M), extracted with diethyl ether. The organic phase was washed with saturated sodium hydrogen carbonate and saturated sodim chloride (aq.). The mixture was dried over sodium sulfate, filtered and evaporated in vacuo. The crude oil was dissolved in a small amount of ethyl acetate and purified by silica chromatography with a gradient of ethyl acetate and hexane. Product showed a single spot in TLC (ethyl acetate/hexane 1:2, Rₑ 0.53).

Yield: 59 mg (43%)

¹H NMR (300 MHz, CDCl₃): δ ppm 2.47 (s, 3H) 2.48-2.62 (m, 4H) 5.02-5.09 (m, 1H) 5.10-5.29 (dm, 1H) 7.36 (d, 2H) 7.79 (d, 2H)

¹⁹F NMR (376 MHz, CDCl₃) δ ppm −178.83

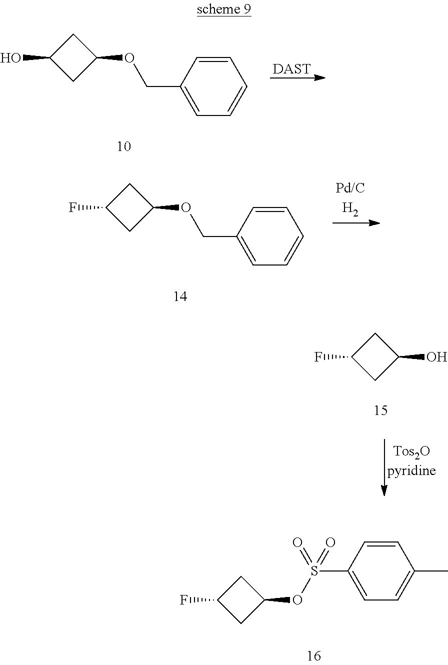

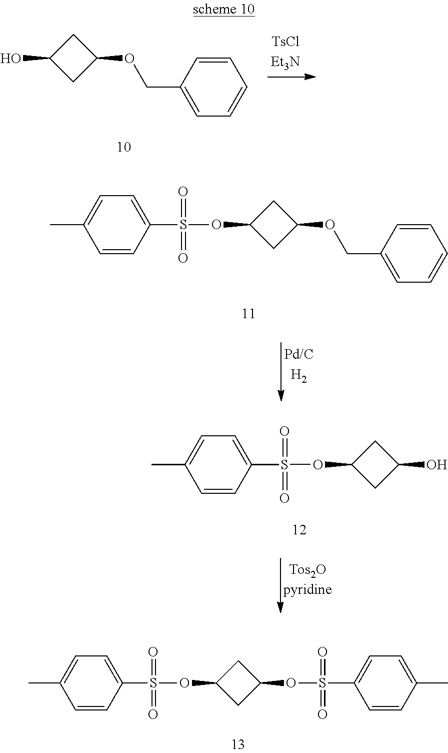

### 1.4 Synthesis of 2-{2-[4-(Cyclobutyloxy)phenyl]-5,7-dimethylpyrazolo[1,5-a]pyrimidin-3-yl}-N,N-diethylacetamide (17)

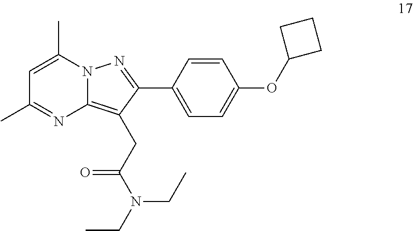

To 14.8 mg (42.56 mmol) of N,N-diethyl-2-[2-(4-hydroxyphenyl)-5,7-dimethylpyrazolo[1,5-a]pyrimidin-3-yl]acetamide in 2 mL dry N,N-dimethylformamide under nitrogene was added 7 mg sodium hydride and stirred for 5 minutes at 25° C., then 11.3 μL of cyclobutylbromid was added and stirred at 25° C. overnight. To the reaction mixture was added 10 mL of ice water and extracted with dichloromethane (3×10 mL). The combined organic phases were washes with water (10 mL) and saturated sodium chloride solution (aq., 10 mL), dried with sodium sulfate, filtered and evaporated in vacuo. Residue was purified by silica chromatography with 95% dichloromethane/5% methanol, followed by a preparative HPLC purification (ACE 5 um C18 250×10 mm, 50% acetonitrile/water, flow: 3 mL/min flow). Product fraction was collected and dry frozen overnight what gave a white solid.

Yield: 10 mg (57%)

¹H NMR (400 MHz, CDCl₃): δ ppm 1.12 (t, 3H) 1.21 (t, 3H) 1.66-1.78 (m, 1H) 1.83-1.94 (m, 1H) 2.12-2.26 (m, 2H) 2.43-2.52 (m, 2H) 2.54 (s, 3H) 2.74 (s, 3H) 3.42 (q, 2H) 3.51 (q, 2H) 3.91 (s, 2H) 4.70 (quin, 1H) 6.51 (s, 1H) 6.90 (d, 2H) 7.74 (d, 2H)

### 1.5 Cold synthesis of N-(2-{4-[4-(Benzyloxy)phenyl]piperazin-1-yl}-2-oxoethyl)-3-[(3-fluorocyclobutyl)oxy]benzamide (24) and the precursor (23) for [¹⁸F]-labeling Benzyl 2-(3-acetoxybenzoylamino)acetate (18)

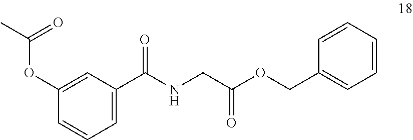

30.4 g (90 mmol) Glycine benzylester p-toluene sulfonate salt are solved in the two phase system dichloromethane and aqueous saturated sodium hydrogencarbonate solution. The organic phase is dried over magnesium sulfate and then evaporated.

13.09 g (79.25 mmol) of free amine is obtained which is used in the subsequent coupling reaction without further purification.

To a solution of 14.28 g (79.25 mmol) 3-Acetoxybenzoic acid in 150 mL tetrahydrofurane and 11 mL triethyl amine (79.25 mmol) at −15° C., 11.39 ml (87.2 mmol) isobutyl chloroformate are added dropwise and the solution is maintained at this temperature for another 15 min. Then, 13.09 g of glycine benzyl ester and 11 mL triethyl amine (79.25 mmol) in 50 mL tetrahydrofurane and 50 mL dichloromethane are added slowly to this cold solution, the temperature is kept below 10° C. for another 15 min and is then allowed to reach room temperature. After stirring overnight the solvent is evaporated and the residue is chromatographed on silica gel using an ethyl acetate/ethanol gradient.

### Yield: 24.6 g (95%).

### 2-(3-Acetoxybenzoylamino)-acetic acid (19)

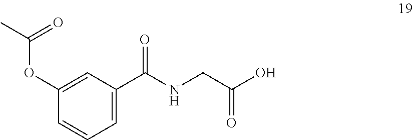

To a solution of 19.64 g (60 mmol) of (3-Acetoxybenzoylamino)-acetic acid benzyl ester (18) in 300 mL methanol was added 3 g Pd on charcoal (10%) and the suspension was stirred under hydrogen overnight at room temperature. The catalyst was filtered off and the solvent evaporated.

Yield: 14.2 g (quantitative).

### Benzyl 4-piperazin-1-ylphenyl ether (20)

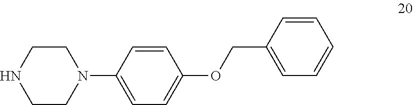

All glassware was dried at 100° C. To a solution of 4.32 g (50.16 mmol) of piperazine in 60 mL toluene were added 459 mg (0.5 mmol) of tris(dibenzylidene acetone)dipalladium(0) and 423 mg (0.68 mmol) of BINAP (2,2′-bis(diphenylphosphino)-1,1′-binaphthyl). Then, a solution of 12 g (45.6 mmol) of 4-benzyloxy-bromobenzene in 40 mL tetrahydrofurane was added followed by a suspension of 6.56 g (68.27 mmol) of sodium t-butylate in tetrahydrofurane.

The reaction mixture was refluxed for 3 hours and stirred at room temperature overnight. After evaporation of the solvents the residue was chromatographed on silica gel using a dichloromethane/methanol gradient.

Yield: 12.2 g (45.7%).

### 3-[N-(2-{4-[4-(Benzyloxy)phenyl]piperazin-1-yl}-2-oxoethyl)carbamoyl]phenyl acetate (21)

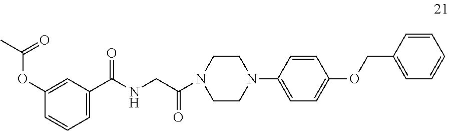

To a solution of 654 mg (2.76 mmol) (3-acetoxybenzoylamino)acetic acid (19) in 70 mL tetrahydrofurane and 0.40 ml triethyl amine (2.87 mmol) at −15° C., 0.396 mL (3.03 mmol) isobutyl chloroformate were added dropwise and the solution was maintained at this temperature for another 15 min. Then, 740 mg of 1-(4-benzyloxyphenyl)piperazine (20) and 1.7 mL triethyl amine (12.25 mmol) in 30 ml tetrahydrofurane and 30 ml dichloromethane were added slowly to this cold solution, the temperature was kept below 10° C. for another 15 min and was then allowed to reach room temperature. After stirring overnight the solvent was evaporated and the residue was chromatographed on silica gel using a hexane/ethyl acetate gradient.

Yield: 390 mg (30%).

### N-(2-{4-[4-(Benzyloxy)phenyl]piperazin-1-yl}-2-oxoethyl)-3-hydroxybenzamide (22)

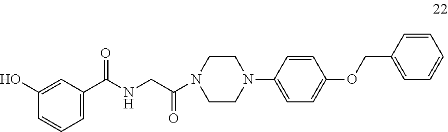

230 mg (0.47 mmol) of the acetate acetic acid 3-{2-[4-(4-benzyloxyphenyl)piperazin-1-yl]-2-oxoethylcarbamoyl}phenyl ester (21) were solved in 30 mL of ethanol and cooled to 0° C. After addition of 1.5 mL 3N sodium hydroxide the solution was stirred for 1 h, glacial acetic acid was added until the pH was below pH 7 and the solvents were evaporated. The raw product was crystallized from ethanol.

Yield: 200 mg (95%).

Compound 22 is the precursor for cold [¹⁹F]-labeling wherein hydroxy is replaced by [¹⁹F].

### trans-3-{3-[N-(2-{4-[4-(Benzyloxy)phenyl]piperazin-1-yl}-2-oxoethyl)carbamoyl]phenoxy}cyclobutyl toluene-4-sulfonate (23)

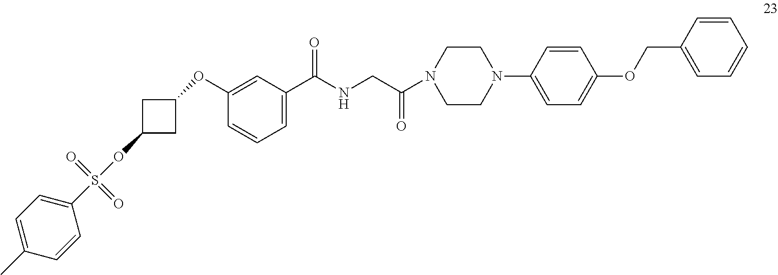

To 111 mg N-{2-[4-(4-Benzyloxyphenyl)piperazin-1-yl]-2-oxoethyl}-3-hydroxybenzamide (22) dissolved in 3 mL N,N-dimethylformamide was added 198 mg (0.5 mmol)

Cyclobutanditosylate and 69 mg (0.5 mmol) potassium carbonate. The reaction mixture was heated in a microwave (high) at 100° C. for 90 min. The solvents were evaporated and the crude product was purified by flash chromatography (dichloromethane/methanol).

Yield: 65 mg (39%).

Compound 23 is the precursor for hot [¹⁸F]-labeling wherein Tosylate is replaced by [¹⁸F].

### N-(2-{4-[4-(Benzyloxy)phenyl]piperazin-1-yl}-2-oxoethyl)-3-[cis-(3-fluorocyclobutyl)oxy]benzamide (24)

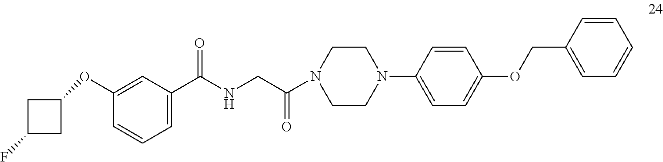

To 60 mg (0.09 mmol) of N-{2-[4-(4-benzyloxyphenyl)-piperazin-1-yl]-2-oxoethyl}-3-(3-toluenesulfonyloxycyclobutyloxy)benzamide dissolved in 3 mL tetrahydrofurane was added 63 mg (0.2 mmol) of tetrabutylammonium fluoride trihydrate. The reaction mixture was heated in a microwave (normal) at 100° C. for 90 min. Another portion of 63 mg (0.2 mmol) of tetrabutylammonium fluoride trihydrate was added and heated in a microwave (normal) at 100° C. for 30 min The reaction mixture was diluted with ethyl acetate and washed with water, dried over sodium sulfate, filtered and the solvents were evaporated. The crude product was purified by flash chromatography (hexane/ethylacetate).

Yield: 12 mg 26%).

### 1.6 Cold Synthesis of [¹⁹F]-Fluoro Labeled Tyrosine and Precursors for Direct Labeling, Scheme 11

### Methyl O-[trans-3-(benzyloxy)cyclobutyl]-N-(tert-butoxycarbonyl)-L-tyrosinate (25)

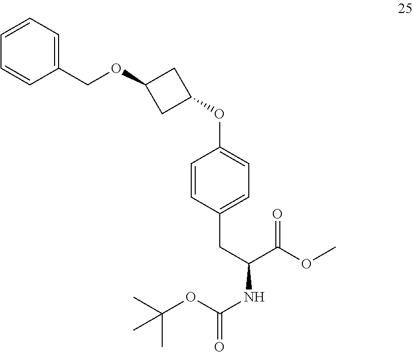

To a solution of 1.02 g (3.35 mmol) of Boc-Tyr-OMe and 1.327 g (7.37 mmol) of cis-3-(benzyloxy)cyclobutanol (10) in 25 mL dry N,N-dimethylformamide 1.197 mL (7.37 mmol) of diethyl azodicarboxylate was added. The yellow solution was stirred for 5 min under nitrogen atmosphere, then 1.975 g (7.37 mmol) of triphenylphosphine was added. The mixture was stirred under nitrogen at 25° C. for 23 h and concentrated under reduced pressure at 80° C. and 19 mbar. The crude oil was dissolved in 50 mL chloroform and washed 3× with 30 mL water to remove N,N-dimethylformamide. The organic layer was dried with anhydrous sodium sulfate, filtered and the solvent was concentrated in vacuo to give 5.556 g of a brown oil. The crude product was purified by silica chromatography with a gradient of ethyl acetate and hexane. Product showed a single spot in TLC (ethyl acetate/hexane 1:2, R_{f}˜0.46).

Yield: 1.35 g (88%)

¹H NMR (400 MHz, CDCl₃): δ ppm 1.43 (s, 9H) 2.38-2.56 (m, 4H) 2.94-3.11 (m, 2H) 3.72 (s, 3H) 4.30-4.39 (m, 1H) 4.46 (s, 2H) 4.50-4.60 (m, 1H) 4.78-4.88 (m, 1H) 4.90-5.00 (m, 1H) 6.71 (d, 2H) 7.02 (d, 2H) 7.29-7.42 (m, 5H)

### Methyl N-(tert-butoxycarbonyl)-O-(trans-3-hydroxycyclobutyl)-L-tyrosinate (26)

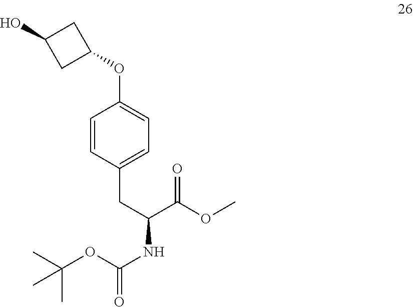

A solution of 1.346 g (2.96 mmol) of methyl O-[trans-3-(benzyloxy)cyclobutyl]-N-(tert-butoxycarbonyl)-L-tyrosinate (25) in 20 mL methanol was stirred with 500 mg 10% palladium on charcoal (50% wet). The mixture was stirred under a positive pressure of hydrogen at 25° C. The mixture was filtered and the solvent evaporated. The oil was dissolved in dichloromethane, filtered through Celite, washed with dichloromethane and evaporated under reduced pressure. Product showed a single spot in TLC (ethyl acetate, R_{f}˜0.54).

Yield: 1.02 g (93%)

¹H NMR (300 MHz, CDCl₃): δ ppm 1.42 (s, 9H) 1.80 (br. s., 1H) 2.34-2.59 (m, 4H) 3.02 (m, 2H) 3.72 (s, 3H) 4.47-4.59 (m, 1H) 4.60-4.70 (m, 1H) 4.79-4.90 (m, 1H) 4.96 (d, 1H) 6.71 (d, 2H) 7.02 (d, 2H)

### Methyl N-(tert-butoxycarbonyl)-O-(cis-3-fluorocyclobutyl)-L-tyrosinate (27)

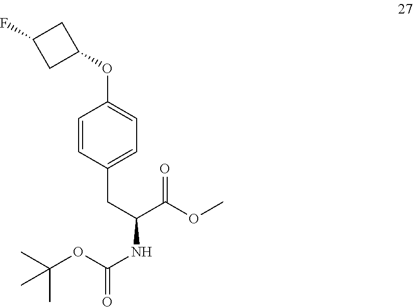

658 mg (1.80 mmol) of methyl N-(tert-butoxycarbonyl)-O-(trans-3-hydroxycyclobutyl)-L-tyrosinate (26) was dissolved in 25 mL dry dichloromethane by stirring under nitrogen. The solution was cooled to 0° C. with an ice bath and 358 μl, (2.70 mmol) of diethylaminosulfur trifluoride was added. The mixture was stirred for 3 h at 0° C. and than let reach room temperature overnight.

The crude product was purified by column chromatography with a gradient of ethyl acetate and hexane. Product showed a single spot in TLC (ethyl acetate/hexane 1:2, R_{f}˜0.62).

Yield: 257 mg (38%)

¹H NMR (300 MHz, CDCl₃): δ ppm 1.42 (s, 9H) 2.36-2.53 (m, 2H) 2.95-3.08 (m, 4H) 3.72 (s, 3H) 4.17-4.27 (m, 1H) 4.50-4.60 (m, 1H) 4.73-5.02 (m, 2H) 6.73 (d, 2H) 7.03 (d, 2H)

¹⁹F NMR (376 MHz, CDCl₃): δ ppm=−169.27 8 (m)

### N-(tert-butoxycarbonyl)-O-(cis-3-fluorocyclobutyl)-L-tyrosine (28)

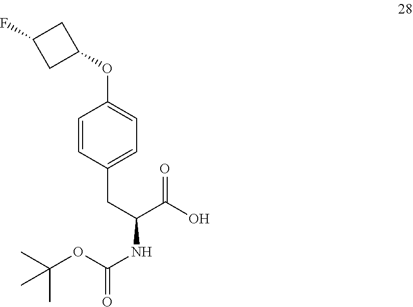

To a solution of 11 mg (30.8 μmol) of methyl N-(tert-butoxycarbonyl)-O-(cis-3-fluorocyclobutyl)-L-tyrosinate (27) in 1 mL methanol 100 μL 1M lithium hydroxide was added. The clear mixture was stirred at 25° C. for 6 h. TLC showed full conversion. With 80 μL 1M hydrochloric acid the mixture was neutralized and evaporated. The resulting oil was dissolved in ethyl acetate. The mixture was washed with saturated sodium chloride (aq.) and evaporated to dryness, was re-dissolved in ethyl acetate, dried over sodium sulfate, filtered and evaporated to give 10 mg.

Yield: 10 mg (92%)

¹H NMR (300 MHz, CDCl₃): δ ppm 1.29-1.49 (m, 9H) 2.33-2.51 (m, 2H) 2.92-3.18 (m, 4H) 4.16-4.27 (m, 1H) 4.52 (d, J=5.56 Hz, 1H) 4.83 (d, J=56.08 Hz, 1H) 4.99 (d, J=7.58 Hz, 1H) 6.05 (br. s., 0H) 6.74 (d, J=8.34 Hz, 2H) 7.09 (d, J=8.34 Hz, 2H)

¹⁹F NMR (376 MHz, CDCl₃): δ ppm=−169.22 (m)

### O-(cis-3-Fluorocyclobutyl)-L-tyrosine trifluoroacetate salt (FCBT) (29)

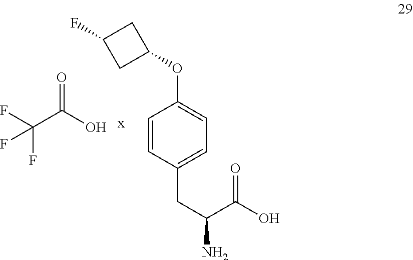

9 mg (25.4 μmol) of N-(tert-butoxycarbonyl)-O-(cis-3-fluorocyclobutyl)-L-tyrosine (28) was dissolved in dichloromethane and treated with 100 μL 25% (v/v) trifluoroacetic acid in dichloromethane for 1 h at 25° C. The mixture was evaporated and provided 7.5 mg of a white solid.

Yield: 7 mg (73%)

¹H NMR (300 MHz, MeOD): δ ppm 2.19-2.36 (m, 2H) 2.95-3.12 (m, 3H) 3.24 (dd, 1H) 4.16 (dd, 1H) 4.27-4.37 (m, 1H) 4.77 (quin, 1H) 6.85 (d, 2H) 7.20 (d, 2H)

¹⁹F NMR (376 MHz, MeOD): δ ppm=−170.43 (m)

### Methyl N-(tert-butoxycarbonyl)-O-[trans-3-(tosyloxy)cyclobuty]-L-tyrosinate (30a)

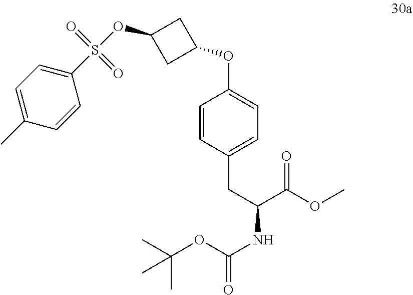

145.3 mg (0.40 mmol) of methyl N-(tert-butoxycarbonyl)-O-(trans-3-hydroxycyclobutyl)-L-tyrosinate (26) was dissolved in 16 mL dry dichloromethane under nitrogen and cooled to 0° C. To this 62.9 mg (0.80 mmol) of pyridine was added followed by 194.7 mg (0.60 mmol) of p-toluenesulfonic anhydride. The mixture was stirred for 5 hours at 0° C. under a nitrogen atmosphere allowed to reach 25° overnight. The mixture was concentrated under reduced pressure and purified by silica chromatography with a gradient of ethyl acetate and hexane. Product showed a single spot in TLC (ethyl acetate/hexane 1:1, R_{f}˜0.58).

Yield: 162 mg (78%)

¹H NMR: (400 MHz, CDCl₃) δ ppm 1.42 (s, 9H) 2.44-2.54 (m, 5H) 2.49-2.66 (m, 2H) 3.01 (qd, 2H) 3.71 (s, 3H) 4.49-4.58 (m, 1H) 4.79 (tt, 1H) 4.94 (d, 1H) 5.00-5.09 (m, 1 H) 6.64 (d, 2H) 7.00 (d, 2H) 7.36 (d, 2H) 7.80 (d, 2H)

Compound 30a is the precursor for hot [¹⁸F]-labeling wherein Tosylate is replaced by [¹⁸F].

### Methyl N-(tert-butoxycarbonyl)-O-[cis-3-(tosyloxy)cyclobutyl]-L-tyros (30b)

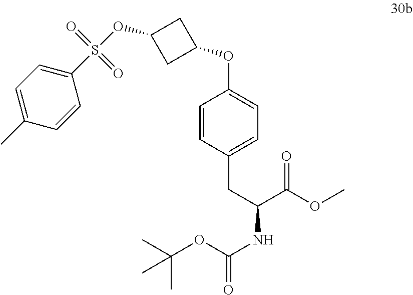

100 mg (0.27 mmol) of methyl N-(tert-butoxycarbonyl)-O-(trans-3-hydroxycyclobutyl)-L-tyrosinate (26) and 137 mg (0.54 mmol) of pyridine 4-methylbenzenesulfonate (PPTS) were dissolved in 2 mL dry tetrahydrofurane and stirred under nitrogen. 143 mg (0.54 mmol) triphenylphosphine was added as a solution in 1 mL tetrahydrofurane and the mixture was cooled in an ice bath. 86 μL (0.54 mmol) diethyl azodicarboxylate was added to the mixture and was stirred for 10 minutes at 0° C., then overnight at 25° C. The suspension was diluted with ethyl acetate, washed with saturated sodium hydrogen carbonate and saturated sodium chloride (aq.). The organic phase was dried with sodium sulfate, filtered and evaporated to dryness. The crude oil was dissolved in a small amount of ethyl acetate and purified by column chromatography with a gradient of ethyl acetate and hexane. Product showed a single spot in TLC (ethyl acetate/hexane 2:1, R_{f}˜0.59).

Yield: 34 mg (24%)

¹H NMR: (300 MHz, CDCl₃) δ ppm 1.41 (s, 9H) 2.34 (dtd, 2H) 2.46 (s, 3H) 2.85 (dtd, 2H) 2.91-3.10 (m, 2H) 3.70 (s, 3H) 4.21 (quin, 1H) 4.47-4.68 (m, 2H) 4.95 (d, 1H) 6.65 (d, 2 H) 7.00 (d, 2H) 7.35 (d, 2H) 7.80 (d, 2H)

Compound 30b is the precursor for hot [¹⁸F]-labeling wherein Tosylate is replaced by [¹⁸F].

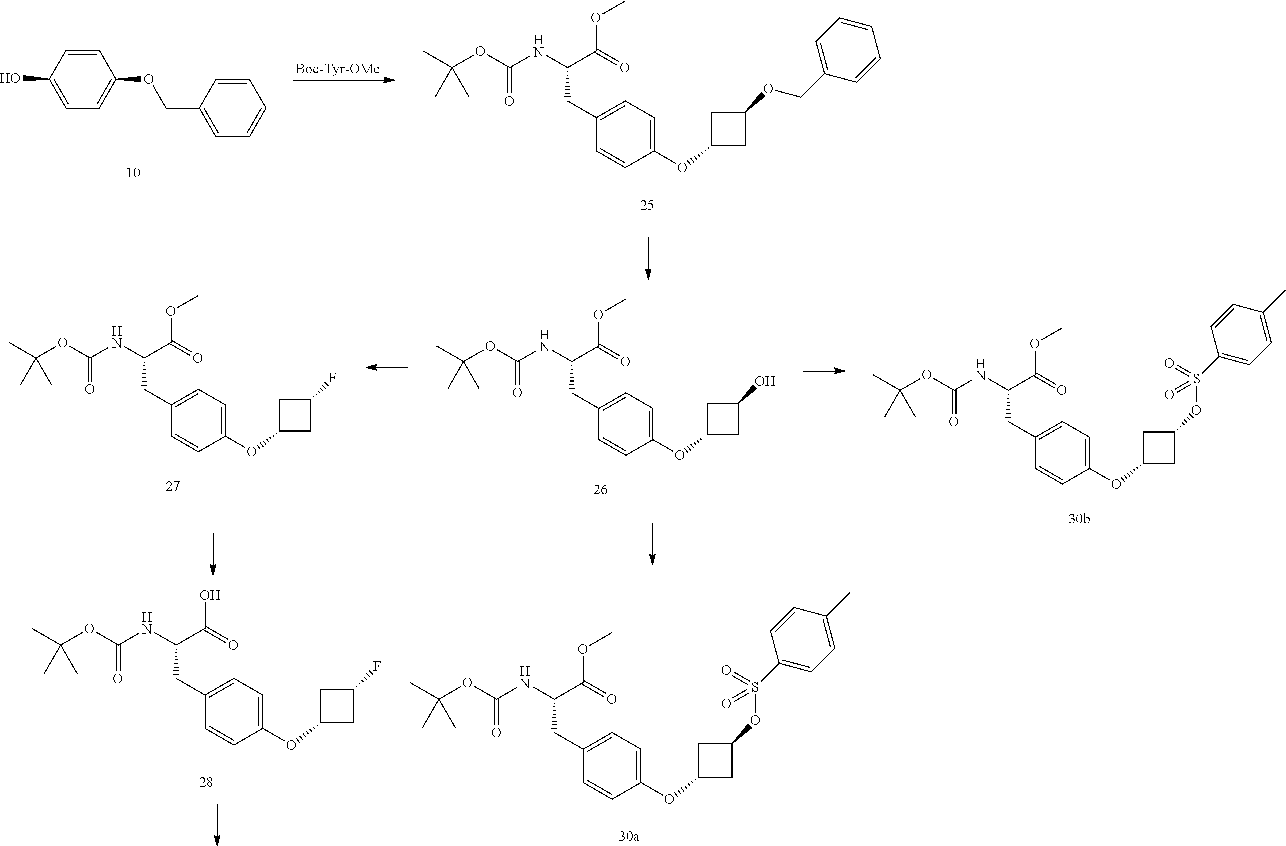

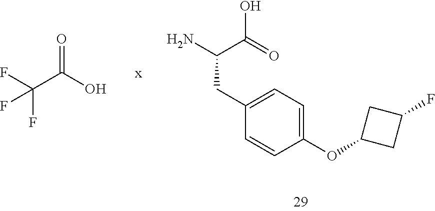

### 2. Experimental

### Radiochemistry

### 2.1 [¹⁸F]Fluoro Labeled Tyrosine

### Indirect method

### 3-[¹⁸F]Fluorocyclobutyl toluene-4-sulfonate (31)

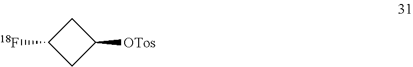

In radiofluorination [¹⁸F]Fluoride (705 MBq) was eluted from a QMA cartridge (equilibrated with 1 M sodiumbicarbonate, washed with 10 mL water) with 2 mL of 0.14 mL water/0.86 mL acetonitrile containing 5 mg Kryptofix (K₂₂₂) and 1.8 mg potassium carbonate into a reaction vial. The solvents were evaporated and the residue dried at 90° C. under a light N₂-stream, more acetonitrile was added, and the drying process was repeated. Precursor cis-Cyclobutyl bis-(4-methylbenzenesulfonate (13), 5 mg) in 500 μL acetonitrile was added to the reaction vial, the reaction stirred for 20 min at 130° C. The crude product was purified by passing through a Waters C18 light (equilibrated with 5 mL ethanol, 5 mL water), washing with 3 mL water and eluted with 1 mL acetonitrile or 1 mL dimethyl sulphoxide. The reaction mixture and isolated product were analyzed by radioTLC and radio-HPLC. The radiochemical yield was 40% (decay corrected) and the radiochemical purity was greater than 99%. Compound 31 is the [¹⁸F]-intermediate for the synthesis of compound 32

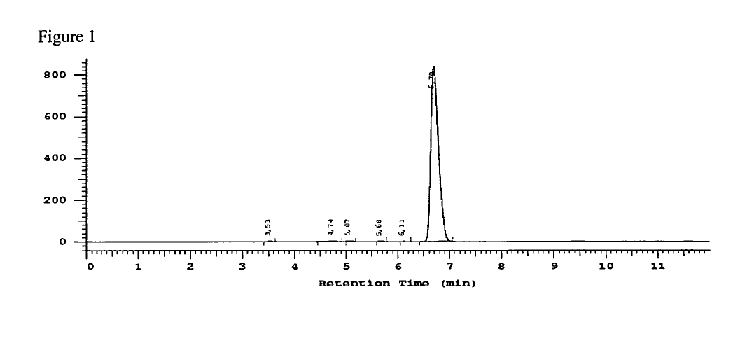

FIG. 1 shows a chromatogram (radio trace) of purified toluene-4-sulfonic acid 3-[¹⁸F]fluoro-cyclobutyl ester (31) and below table 1 accompanying the chromatogram.

TABLE 1
Radio trace

| No. | RT | Area | Conc 1 | BC |
|---|---|---|---|---|
| 1 | 3.53 | 14441 | 0.164 | BB |
| 2 | 4.74 | 32558 | 0.369 | BB |
| 3 | 5.07 | 14030 | 0.159 | BB |
| 4 | 5.68 | 11469 | 0.130 | BB |
| 5 | 6.11 | 7047 | 0.080 | BB |
| 6 | 6.70 | 8746287 | 99.099 | BB |
| | | 8825832 | 100.000 | |

### Sodium O-(cis-3-[¹⁸F]fluorocyclobutyl)-L-tyrosinate (32a) (indirect method 1)

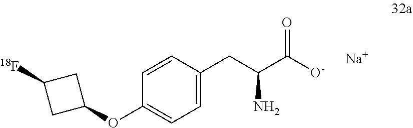

The toluene-4-sulfonic acid 3-[¹⁸F]fluorocyclobutyl ester (31) in dimethyl sulphoxide (1 mL) was added to a solution of L-Tyrosine disodium salt (J. Nuc. Med., 1999, 40, p 205, 7 mg) and stirred for 15 min at 150° C. The reaction mixture was purified by semi-preparative HPLC(C-18 reversed phase column acetonitrile/water=45/55, flow=4 mL/min). The resulting product was analyzed by radio-HPLC and confirmed by co-injection. The product was isolated with a radiochemical purity of more than 91%.

### O-(cis-3-[¹⁸F]Fluorocyclobutyl)-L-tyrosine (32b) (indirect method 2)

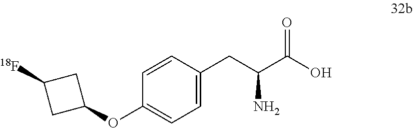

The toluene-4-sulfonic acid 3-[¹⁸F]Fluorocyclobutyl ester (31) in dimethyl sulphoxide (1 mL) was added to a solution of L-Tyrosine (5 mg) in 22.1 μL 10% sodium hydroxide (aq). The reaction was heated at 150° C. for 10 min. To the reaction mixture was added 15 mL water pH 2 and purified by HPLC (Synergi Hydro RP 4μ 250×10 mm; 15% acetonitrile in water at pH 2; flow 3 mL/min). The product peak was collected, diluted with water (pH 2) and passed through a C18 SPE (preconditioned by washing the cartridge with 5 mL ethanol and 10 mL water). The SPE was washed with water pH2 (5 mL). The product was eluted with a 1:1 mixture of ethanol and water pH 2 (1.5 mL). Starting from 881 MBq [¹⁸F]fluoride, 44 MBq (12% d.c.) of desired product were obtained in 144 min. The product was analyzed by radio-HPLC (ACE 3 C18 50×4.6 mm; solvent A. water+0.1%; solvent B: acetonitrile+0.1% trifluoroacetic acid: gradient 5% B to 95% B in 7 min) and the desired product peak (RT=2.768 min) was observed and confirmed by injection of reference compound.

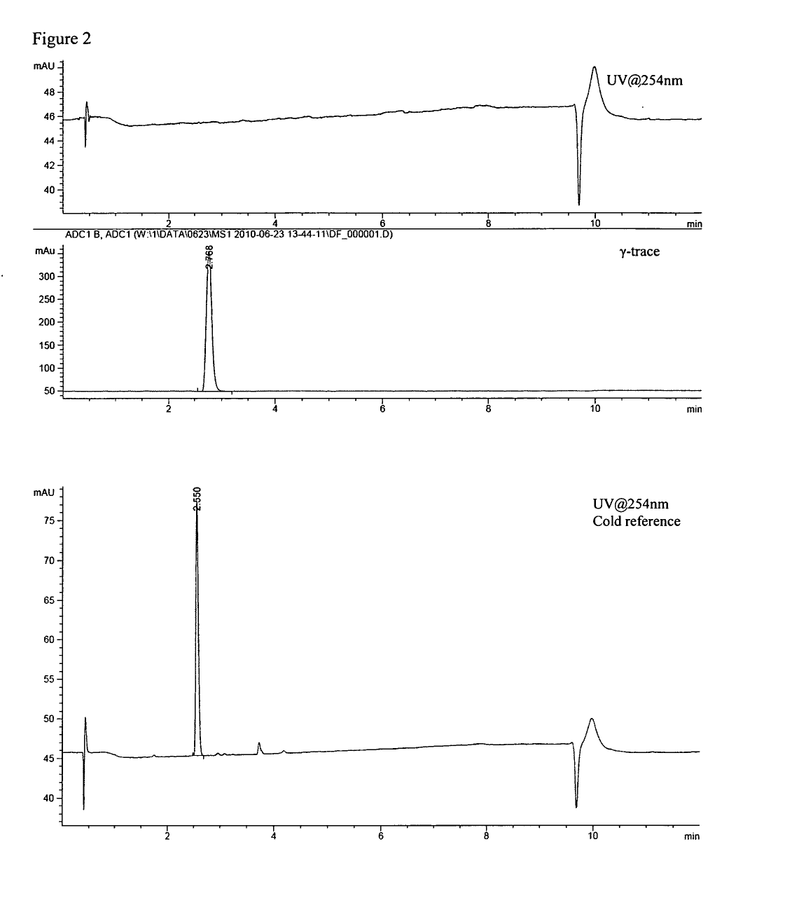

FIG. 2 shows a chromatogram (radio trace) of purified (S)-2-Amino-3-[4-(3-[¹⁸F]fluoro-cyclobutoxy)-phenyl]-propionic acid (32b) compared to the cold reference and below tables 2 and 3 accompanying the chromatograms

TABLE 2

| Peak | RetTime | | Width | Area | Height | Area |
|---|---|---|---|---|---|---|
| # | [min] | Type | [min] | [mAu*s] | [mAu] | % |
| 1 | 2.768 | VV | 0.1155 | 2198.86230 | 303.69061 | 100.0000 |

TABLE 3

| Peak | RetTime | | Width | Area | Height | Area |
|---|---|---|---|---|---|---|
| # | [min] | Type | [min] | [mAu*s] | [mAu] | % |
| 1 | 2.550 | MM | 0.0547 | 104.99688 | 31.97873 | 100.0000 |

### 2.2 [¹⁸F]Fluoro Labeled Tyrosine Direct Method

### Methyl N-(tert-butoxycarbonyl)-O-(cis-3-[¹⁸F]fluorocyclobutyl)-L-tyrosinate (33)

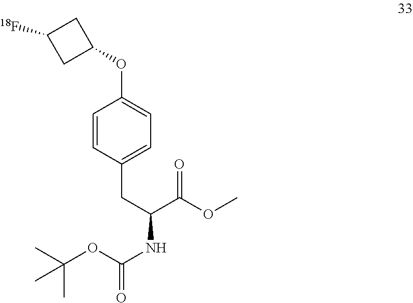

In radiofluorination [¹⁸F]Fluoride (668 MBq) was eluted from a QMA cartridge (equilibrated with 0.5 M potassium carbonate, washed with 10 mL water) with 2 mL of 0.05 mL water/0.95 mL acetonitrile containing 5 mg Kryptofix (K₂₂₂) and 1 mg potassium carbonate into a reaction vial. The solvents were evaporated and the residue dried at 90° C. under a light N₂-stream, more acetonitrile was added, and the drying process was repeated. Precursor (methyl N-(tert-butoxycarbonyl)-O-(trans-3-{[(4-methylphenyl)sulfonyl]oxy}cyclobutyl)-L-tyrosinate (30a), 3 mg) in 500 μL acetonitrile was added to the reaction vial, the reaction stirred for 10 min at 110° C. The reaction mixture was analyzed by radio-HPLC where the desired product peak (RT=5.274) could be observerd and confirmed by injection of reference compound.

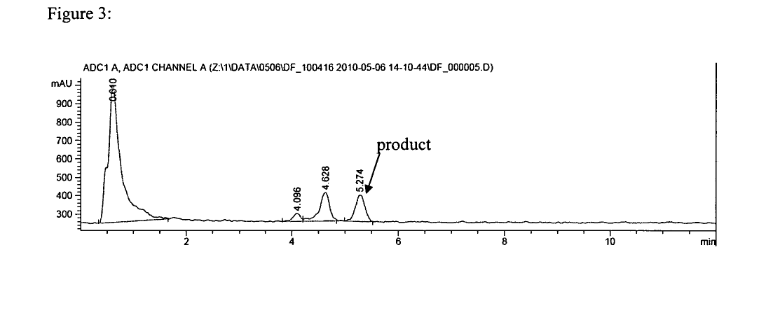

FIG. 3 shows a chromatogram (radio trace) of reaction mixture of methyl N-(tert-butoxycarbonyl)-O-(cis-3-fluorocyclobutyl)-L-tyrosinate (33) and below table 4 accompanying the chromatograms

TABLE 4

| Peak | RetTime | | Width | Area | Height | Area |
|---|---|---|---|---|---|---|
| # | [min] | Type | [min] | [mAU*s] | [mAU] | % |
| 1 | 0.610 | MM | 0.2895 | 1.29692e4 | 746.62274 | 75.1762 |
| 2 | 4.096 | MF | 0.1684 | 430.94138 | 42.66048 | 2.4979 |
| 3 | 4.628 | FM | 0.2226 | 2061.41284 | 154.35629 | 11.9490 |
| 4 | 5.274 | MM | 0.2092 | 1790.20569 | 142.60684 | 10.3769 |

| | | |
|---|---|---|
| Totals: | 1.72518e4 | 1086.24636 |

### 2.3 Synthesis of (cis)-Methyl 3-[¹⁸F]fluorocyclobutaneearboxylate—Fluorolabeling (cis)-Methyl 3-[¹⁸F]fluorocyclobutaneearboxylate (34)

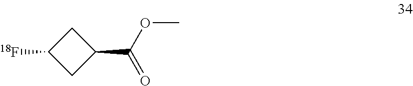

In radiofluorination [¹⁸F]Fluoride (483 MBq) was eluted from a QMA cartridge (equilibrated with 1 M sodium hydrogen carbonate, washed with 10 mL water) with 1 mL of 0.14 mL water/0.86 mL acetonitrile containing 5 mg Kryptofix (K₂₂₂) and 1.8 mg potassium carbonate into a reaction vial. The solvents were evaporated and the residue dried at 90° C. under a light N₂-stream, more acetonitrile was added, and the drying process was repeated. Precursor cis-Methyl 3-(4-methylbenzenesulfonyl)cyclobutanecarboxylate (3), 5 mg) in 500 μL dimethyl sulphoxide was added to the reaction vial, the reaction stirred for 20 min at 125° C. The reaction mixture was analyzed by radioTLC and radio-HPLC. The radiochemical yield was 43% (decay corrected).

### 2.4 Synthesis of trans-3-[¹⁸F]fluorocyclobutanecarboxylic acid (36)—Fluorolabeling trans-Benzyl 3-[¹⁸F]fluorocyclobutanecarboxylate (35)

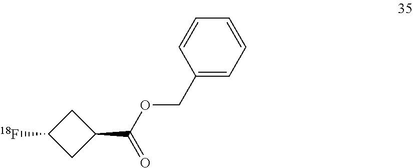

In radiofluorination [¹⁸F]Fluoride (1385 MBq) was eluted from a QMA cartridge (equilibrated with 0.5 M potassium carbonate, washed with 10 mL water) with 1 mL of 0.05 mL water/0.95 mL acetonitrile containing 5 mg Kryptofix (K₂₂₂) and 1. mg potassium carbonate into a reaction vial. The solvents were evaporated and the residue dried at 90° C. under a light N₂-stream, more acetonitrile was added, and the drying process was repeated. Precursor cis-Benzyl 3-(4-methylbenzenesulfonyl)cyclobutanecarboxylate (8), 4.7 mg) in 500 μL dimethyl suphoxide was added to the reaction vial, the reaction stirred for 10 min at 180° C. The product was analyzed by HPLC and radioTLC. The product was confirmed by co-injection with the reference compound.

The crude product was purified by passing through a Waters C18 light (equilibrated with 5 ml ethanol, 5 mL water), washing with 3 mL water and eluted with 1 mL acetonitrile. The reaction mixture and isolated product were analyzed by radioTLC and radio-HPLC. The two isomers were separated by semi-preparative HPLC(C18 reversed phase column acetonitrile/water=55/45, flow=3 mL/min). The product fraction (confirmed by co-injection) was diluted with 30 mL water and loaded onto a equilibrated Waters C18 cartridge and eluted with 1 mL ethanol what was used without further purification.

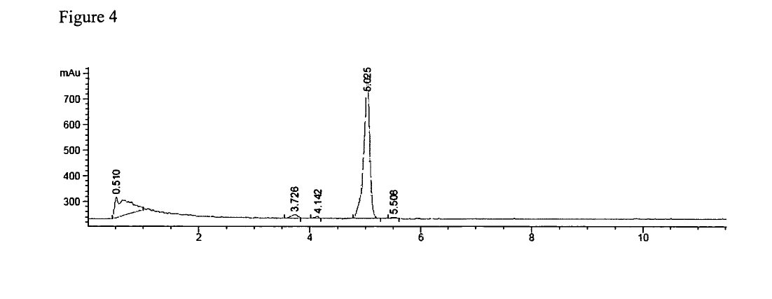

FIG. 4 shows a chromatogram (radio trace) of trans-benzyl 3-[¹⁸F]fluorocyclobutane-carboxylate (35) and table 5.

TABLE 5

| Peak | RetTime | | Width | Area | Height | Area |
|---|---|---|---|---|---|---|
| # | [min] | Type | [min] | [mAu*s] | [mAu] | % |
| 1 | 0.510 | MM | 0.2757 | 1329.02246 | 80.35561 | 23.4810 |
| 2 | 3.726 | MM | 0.1274 | 112.84705 | 14.76173 | 1.9938 |
| 3 | 4.142 | MM | 0.0739 | 32.09610 | 7.23672 | 0.5671 |
| 4 | 5.025 | MM | 0.1234 | 4159.67383 | 561.67389 | 73.4927 |
| 5 | 5.508 | MM | 0.0953 | 26.34429 | 4.60600 | 0.4654 |

### trans-3-[¹⁸F]fluorocyclobutanecarboxylic acid (36)

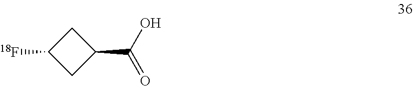

trans-Benzyl 3-[¹⁸F]fluorocyclobutanecarboxylate (35) in 1 mL ethanol was treated with 1.0 mL 1M sodium hydroxide for 5 min at 25° C. and neutralized with 1M hydrochloric acid. The radiochemical yield was 17% (decay corrected) and the radiochemical purity was greater than 99%.

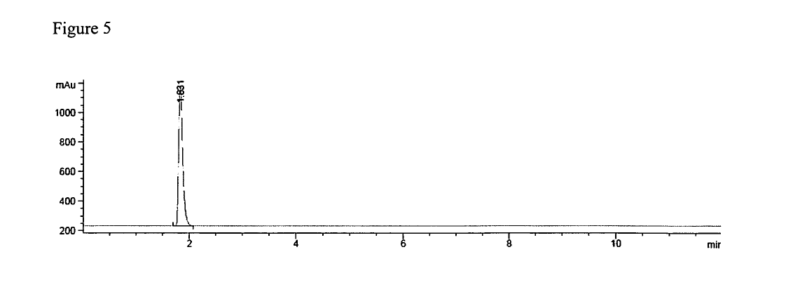

FIG. 5 shows a chromatogram (radio trace) of (trans)-3-[¹⁸F]fluorocyclobutanecarboxylate (36) and table 6.

TABLE 6

| Peak | RetTime | | Width | Area | Height | Area |
|---|---|---|---|---|---|---|
| # | [min] | Type | [min] | [mAu*s] | [mAu] | % |
| 1 | 1.831 | MM | 0.0866 | 4903.75244 | 943.61340 | 100.0000 |

### 2.5 Synthesis of N-(2-{4-[4-(Benzyloxy)phenyl]piperazin-1-yl}-2-oxoethyl)-3-(cis-3-fluorocyclobutyloxy)benzamide—Fluorolabeling N-(2-{4-[4-(Benzyloxy)phenyl]piperazin-1-yl}-2-oxoethyl)-3-(cis-3-[¹⁸F]fluorocyclobutyloxy)benzamide (37)

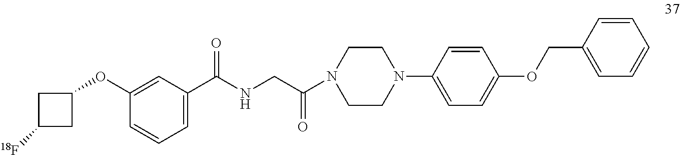

In radiofluorination [¹⁸F]Fluoride (604 MBq) was eluted from a QMA cartridge (equilibrated with 0.5M potassium carbonate, washed with 10 mL water) with 8 μL 40% tetrabutylammonium hydroxide_{(aq)} in 1.5 mL acetonitrile and 500 μL water into a reaction vial. The solvents were evaporated and the residue dried at 120° C. under a light N₂-stream, more acetonitrile was added, and the drying process was repeated. Precursor, N-{2-[4-(4-Benzyloxyphenyl)piperazin-1-yl]-2-oxoethyl}-3-(cis-3-toluene sulfonyloxycyclobutyloxy)-benzamide (23) (2 mg) in 500 μL acetonitrile was added to the reaction vial, the reaction stirred for 15 min at 100° C. The reaction mixture was analyzed by HPLC(C18 reversed phase column using a gradient of 5-95% acetonitrile in water+0.1% trifluoroacetic acid over 7 min, flow=2 mL/min).

### 3. Experimental biology

### 3.1 Uptake

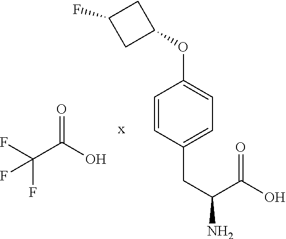

### O-(cis-3-Fluorocyclobutyl)-L-tyrosine trifluoroacetate salt (29) (FCBT)

### Material and Methods:

Cells were seeded 1-2 days prior to the assay and grown until sub-confluency in 48 well plates. Prior to the assay the cell culture medium was removed and the cells were washed with phosphate buffered saline (PBS)+0.1% Bovine serum albumin (BSA). After adding the assay buffer (PBS+0.1% BSA), 37 KBq of the radiotracer [H-3]-D-Tyrosine was added immediately and was incubated with cells at 37° C. in a humidified atmosphere containing 5% CO₂ for 30 min. For investigation of transporter characteristics and competitions, the cells were coincubated with 100 μM F-DOPA or compound 29 (FCBT) for 30 min to monitor radioactivity uptake. To stop tracer uptake the incubation buffer was removed after 30 min, the cells were washed and lysed with 1 M sodium hydroxide. Subsequently the amount of radioactivity in the cell lysate was determined in a scintillation counter.

To study if the compounds are transported by LAT1, the cells were incubated with 37 kBq of the radiotracer [H-3]-D-Tyrosine and was incubated with cells at 37° C. in a humidified atmosphere containing 5% CO₂ for 30 min. Then the cells were washed with PBS and fresh assay buffer was added to the cells containing 100 μM concentration of cold compound and the cells were incubated for another 30 min. To stop tracer efflux the incubation buffer was removed after 30 min, the cells were washed and lysed with 1 M sodium hydroxide. Subsequently the amount of radioactivity in the cell lysate was determined in a scintillation counter.

Aliquots of the applied tracer amount were measured in a gamma counter to determine the total amount in counts per minute (cpm) together with the samples to correct for tracer decay. Cell numbers per well were determined after detaching cells by trypsinization in 3 wells prior to start of the assay and counted in cell chamber under a microscope. The mean of cells was calculated. To compare tracer uptake between different studies the cell number was normalized to 100,000 cells.

### Results

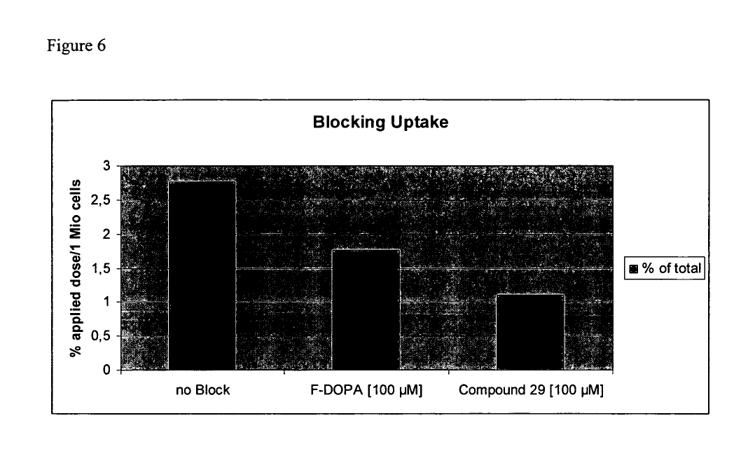

The A549 human lung carcinoma cell line showed 2.8% uptake of the applied [H-3]-D-Tyrosine after 30 min (FIG. 1). This uptake was reduced to 1.8% if F-DOPA was present in the assay buffer and even further reduced to 1.1% if compound 29 (FCBT) was present in the assay buffer. This clearly showed that F-DOPA and compound 29 (FCBT) effectively compete with D-Tyrosine for the uptake into the cells. The exclude that this effect is due to a transporter blocking and not competition for the transport, an efflux experiment was performed. The LAT transporter, which is responsible for the uptake of large aromatic amino acids such as Tyrosine, is an exchanger which transports one amino acid out of the cell for each amino acid it transports into the cell. If compound 29 (FCBT) is indeed a substrate of the LAT transporter it should stimulate the efflux of D-Tyrosine out of the cell. The experiment in FIG. 2 showed an efflux of D-Tyrosine to 0.7% applied dose/100.000 cells after 30 min. Adding F-DOPA increase the efflux of D-Tyrosine and only 0.11% applied dose/100.000 cells remained in the cells after 30 min. The effect of compound 29 (FCBT) was even greater and the amount of D-Tyrosine, which remained in the cell after 30 min was only 0.08% applied dose/100.000 cells, see FIGS. 6 and 7.

### 3.2 Investigation of In Vitro Metabolic Stability in Rat Hepatocytes (Including Calculation of Hepatic In Vivo Blood Clearance (CL))

Hepatocytes from Han Wistar rats were isolated via a 2-step perfusion method. After perfusion, the liver was carefully removed from the rat: the liver capsule was opened and the hepatocytes were gently shaked out into a Petri dish with ice-cold WME. The resulting cell suspension was filtered through sterile gaze in 50 mL falcon tubes and centrifuged at 50×g for 3 min at room temperature. The cell pellet was resuspended in 30 mL WME and centrifuged through a Percoll® gradient for 2 times at 100×g. The hepatocytes were washed again with Williams' medium E (WME) and resuspended in medium containing 5% FCS. Cell viability was determined by trypan blue exclusion.

For the metabolic stability assay liver cells were distributed in WME containing 5% FCS to glas vials at a density of 0.5×10⁶ vital cells/ml. The test compound was added to a final concentration of 1 μM. During incubation, the hepatocyte suspensions were continuously shaken and aliquots were taken at 2, 8, 16, 30, 45 and 60 min, to which equal volumes of cold methanol were immediately added. Samples were freezed at −20° C. overnight, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, amount of liver cells in vivo and in vitro the hepatic in vivo blood clearance (CL) was calculated The following parameter values were used: Liver blood flow—4.2 L/h/kg human; specific liver weight—32 g/kg rat body weight; liver cells in vivo—1.1×10⁸ cells/g liver, liver cells in vitro—0.5×10⁶/mL.

Both compounds are very stable in rat hepatocytes.

TABLE 7

| Compound | Compound 29 | O-(2- |
|---|---|---|
| | | [¹⁹F]Fluoroethyl)-L- |
| | | tyrosine |
| Species | rat | rat |
| Strain | Wistar | Wistar |
| Sex | male | male |
| Hepatocytes, cell no. | 0.5 | 0.5 |
| [106/ml] | | |
| Hep. Viablility (corr.) [%] | 100 | 100 |
| Incubation period [min] | 2-8-16-30-45-90 | 2-8-16-30-45-90 |
| CL blood, ws, hep [L/h/kg] | 0.43 | 0.0001 |

### 3.3 Investigation of In Vitro Plasma Stability

This investigation determines the stability of test compound in plasma of different species. Test compound was incubated in plasma of male rats and female human for different time points (2, 30 and 60) at a concentration of 0.3 μM. Samples were freezed at −20° C. overnight, subsequently centrifuged for 15 minutes at 3000 rpm and the supernatant was analyzed with an Agilent 1200 HPLC-system with LCMS/MS detection.

The stability of the test compound was quantified by comparison of the remaining amount at the different time points with the amount of the 0 min sample and is expressed in % of initial concentration.

Plasma stability testing in rat plasma showed that both compounds were stable in rat plasma for up to 60 min. Whereas in human plasma the reference compound O-(2-[¹⁹F]Fluoroethyl)-L-tyrosine (FET) showed 50% degradation after 60 min while compound 29 did not show any degradation after 60 min.

TABLE 8
Plasma stability of compound 29 and O-(2-[¹⁹F]Fluoroethyl)-L-tyrosine
(FET)
Plasma stability of compound 29 in rat:

| Probe | % v. 0 h | MEAN [%] |
|---|---|---|
| 2 min rat, male Incubation] | 89 | 100 |
| 2 min rat, male Incubation] | 111 | |
| 30 min rat, male Incubation] | | 124 |
| 30 min rat, male Incubation] | 124 | |
| 60 min rat, male Incubation] | 132 | 133 |
| 60 min rat, male Incubation] | 134 | |

### Plasma Stability of Compound 29 in Human:

| Probe | % v. 0 h | MEAN [%] |
|---|---|---|
| 2 min human female Incubation] | 96 | 100 |
| 2 min human female Incubation] | 104 | |
| 30 min human female Incubation] | 121 | 111 |
| 30 min human female Incubation] | 100 | |
| 60 min human female Incubation] | 104 | 104 |
| 60 min human female Incubation] | 105 | |

### Plasma Stability of O-(2-[¹⁹F]Fluoroethyl)-L-Tyrosine (FET) in Rat

| | | |
|---|---|---|
| 1 min rat, male Incubation] | 92 | 100 |
| 1 min rat, male Incubation] | 108 | |
| 30 min rat, male Incubation] | 110 | 109 |
| 30 min rat, male Incubation] | 109 | |
| 60 min rat, male Incubation] | 120 | 119 |
| 60 min rat, male Incubation] | 118 | |

### Plasma Stability of O-(2-[¹⁹F]Fluoroethyl)-L-Tyrosine (FET) in Human

| | | |
|---|---|---|
| 1 min human female Incubation] | 101 | 100 |
| 1 min human female Incubation] | 99 | |
| 30 min human female Incubation] | 101 | 103 |
| 30 min human female Incubation] | 105 | |
| 60 min human female Incubation] | 52 | 50 |
| 60 min human female Incubation] | 49 | |

### 3.4 Compound 17

### In Vitro Binding

### 2-{2-[4-(Cyclobutyloxy)phenyl]-5,7-dimethylpyrazolo[1,5-a]pyrimidin-3-yl}-N,N-diethylacetamide (17)

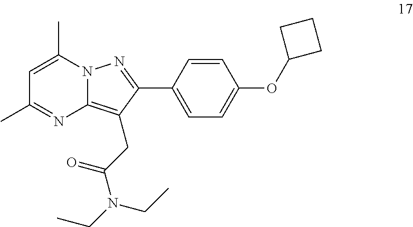 [0351] IC₅₀=4.81 nM
Kᵢ=7.74 •nM
   DPA-714; In Vitro Binding (J. Nuc. Med., 2008, 49, p 814):

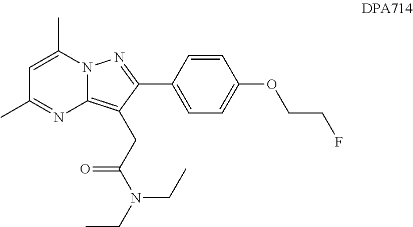

Kᵢ=7.0 •nM

### 3.5 Cell-Uptake Experiments

We studied the uptake of the radiolabeled [¹⁸F] compound 32b into A549 cells, 90000 A549 cells were seeded per cavity of a 48 well incubation plate (Becton Dickinson; Cat. 353078) and incubated for 2 days in RPMI 1640 with GlutaMAX (Invitrogen; Cat. 31331) medium supplemented with 10% FCS in an incubator (37° C., 5% CO₂). Cells were washed once with PBS and then incubated for 10-30 minutes at 37° C. in PBS with 0.25 MBq of compound 32b ([¹⁸F] labeled). After incubation, the cells were washed once with cold PBS, lysed with 1M NaOH, and finally lysates were measured in a gamma counter.

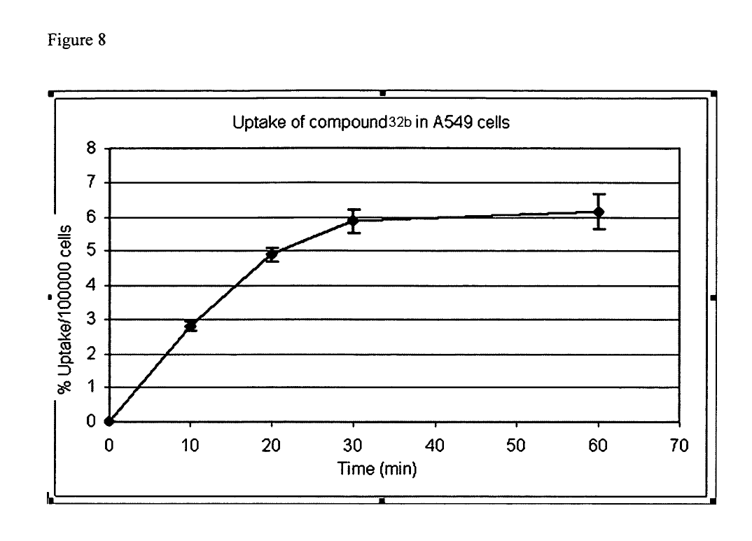

Compound 32b ([¹⁸F] labeled) showed good accumulation in all tested tumor cells. The uptake of compound 32b ([¹⁸F] labelled) increased of time to a maximum of 5.87% applied dose/10⁶ cells in A549 cells after 30 min and remained constant thereafter (see FIG. 8).

### 3.6 Competition Experiments

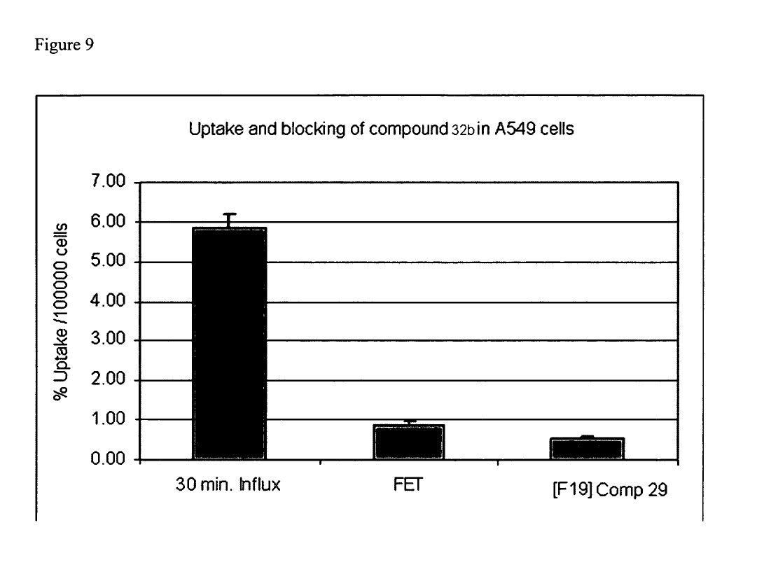

We studied the uptake of the radiolabeled compound 32b ([¹⁸F] labeled) into A549 cells. 100000 A549 cells were seeded per cavity of a 48 well incubation plate (Becton Dickinson; Cat. 353078) and incubated for 2 days in RPMI 1640 with GlutaMAX (Invitrogen; Cat. 31331) medium supplemented with 10% FCS in an incubator (37° C., 5% CO₂). Cells were washed once with PBS and then incubated for 30 minutes at 37° C. in PBS with 0.25 MBq radioactive tracer of compound 32b ([¹⁸F] labeled) plus 1 mM cold [¹⁹F] compound 29 or 1 mM cold FET for competition. After incubation, the cells were washed once with cold PBS, lysed with 1M sodium hydroxide, and finally lysates were measured in a gamma counter. Blocking effect was calculated as percent uptake of blocked compound in comparison to uptake of unblocked compound (see FIG. 9).

1. A compound of Formula I 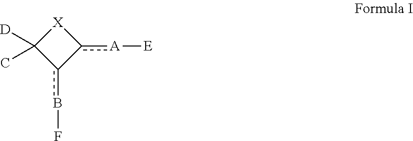
wherein
A=H, —O—, ═O, —S—, ═S, N, N(R′), NYR′, P(R′)(R″), P(O)(R′)R″, C(R′)(R″), CR′R″, C(O), C(O)O, C(O)OR′ C(O)R′R″, SO, SO2, or SO2NR′,
B=H, —O—, ═O, S, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,
Y=N, NR′, O or S,
C=H, Leaving Group (LG), or R′,
D=H, Leaving Group (LG), or R′,
E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′ C(O)R′R″, SO, SO2, SO2NR′, or W-Z, wherein W is a linker and Z is a targeting agent or vector,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′ C(O)R′R″, SO, SO2, SO2NR′, or W-Z, wherein W is a linker and Z is a targeting agent or vector,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
X=(CH₂)_{q} or C(R′R″), and
q=0 to 2,
provided when A or B is O then E or F is absent
or a pharmaceutical salt, diastereomere or enantiomere thereof.

2. The compound according to claim 1 wherein the compound of Formula I is protected at a functional group.

3. The compound according to claim 1 wherein E is not W-Z.

4. A compound of formula II. 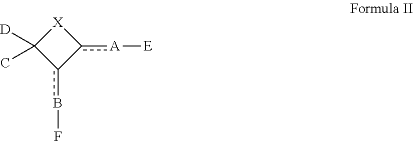
wherein
A=H, —O—, ═O, —S—, ═S, N, N(R′), NYR′, P(R′)(R″), P(O)(R′)R″, C(R′)(R″), CR′R″, C(O), C(O)O, C(O)OR′ C(O)R′R″, SO, SO2, or SO2NR′,
B=H, —O—, ═O, S, ═S, N, N(R′), NYR′, P(R′)(R″), P(O)(R′)R″, C(R′)(R″), CR′R″, C(O), C(O)O, C(O)OR′, C(O)R′R″, SO, SO2, or SO2NR′,
Y=N, NR′, O or S,
Y=N, NR′, O or S,
C=H, radioisotope, halogen or R′,
D=H, radioisotope, halogen or R′,
E=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′ C(O)R′R″, SO, SO2, SO2NR′, or W-Z, wherein W is a linker and Z is a targeting agent,
F=absent, H, OR′, SR′, NR′, CR′ₚ, —O—, ═O, —S—, ═S, N, N(R′), NYR′, P(O)(R′)R″, C(R′)(R″), CR′R″, C(O), C(O)O, C(O)OR′ C(O)R′R″, SO, SO2, SO2NR′, or W-Z, wherein W is a linker and Z is a targeting agent,
p=1 to 3,
R′=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
R″=H, OH, NH, branched or linear C₁-C₆ alkyl, branched or linear O—C₁-C₆ alkyl, branched or linear C₁-C₆ alkoxy, branched or linear C₁-C₆ alkylene, substituted or unsubstituted aryl or substituted or unsubstituted heteroaryl, CO(CH₂)ₙ, [O(CH₂)ₙ—O(CH₂)ₙ]ₘ, or —O(CH₂)ₙ, [O(CH₂)ₙ—O(CH₂)ₙ]ₘ,
n=1 to 6 and m=1 to 6,
X=(CH₂)_{q} or C(R′R″), and
q=0 to 2,
provided when A or B is O then E or F is absent
or a pharmaceutical salt, diastereomere or enantiomere thereof.

5. The compound according to claim 4 wherein the compound of Formula II is protected at a functional group.

6. The compound according to claim 4 wherein E is not W-Z.

7. The compound according to claim 4 wherein C or D is a radioisotope selected from ¹⁸F, ¹²³I, ¹²⁴I, ¹²⁵I, and ¹³¹I.

8. A pharmaceutical composition comprising a compound according to claim 1 and a pharmaceutically acceptable carrier, diluent, excipient or adjuvant.

9. A method for obtaining a compound according to claim 1 comprising the steps
Optionally adding a protecting group to a compound of Formula I wherein C and D are not a Leaving Group,
Reacting the optionally protected compound of Formula I wherein C and D are not a Leaving Group with a Leaving Group to obtain an optionally protected compound of Formula I, and
Optionally unprotecting the compound of Formula I.

10. A method for direct labeling for obtaining a compound according to claim 4 comprising the steps
Optionally adding a protecting group to a compound of Formula I,
Radiolabeling the optionally protected compound of Formula I with a radioisotope to obtain an optionally protected compound of Formula II, and
Optionally unprotecting the compound of Formula II.

11. A method for indirect labeling for obtaining a compound according to claim 4 comprising the steps
Optionally adding a protecting group to a compound of Formula I not containing a targeting agent or vector moiety,
Radiolabeling of the optionally protected compound of Formula I not containing a targeting agent or vector moiety with a radioisotope to obtain an optionally protected compound of Formula II not containing a targeting agent or vector moiety,
Reacting the optionally protected compound of Formula II not containing a targeting agent or vector moiety with a targeting agent or vector moiety to obtain an optionally protected compound of Formula II, and
Optionally unprotecting the compound of Formula II.

12. The compound according to claim 4, wherein C or D is ¹⁸F.

13. A pharmaceutical composition comprising a compound according to claim 2 and a pharmaceutically acceptable carrier, diluent, excipient or adjuvant.

14. A pharmaceutical composition comprising a compound according to claim 3 and a pharmaceutically acceptable carrier, diluent, excipient or adjuvant.

15. A pharmaceutical composition comprising a compound according to claim 4 and a pharmaceutically acceptable carrier, diluent, excipient or adjuvant.

16. A pharmaceutical composition comprising a compound according to claim 5 and a pharmaceutically acceptable carrier, diluent, excipient or adjuvant.

17. A pharmaceutical composition comprising a compound according to claim 6 and a pharmaceutically acceptable carrier, diluent, excipient or adjuvant.
